(19) United States
(12) Patent Application Publication
(10) Pub. No.: US 2004/0071757 A1
(43) Pub. Date: Apr. 15, 2004

(54) Inhalation antiviral patch

(76) Inventor: David Rolf, Eden Prairie, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/458,078

(22) Filed: Jun. 10, 2003

Related U.S. Application Data
(63) Continuation-in-part of application No. 10/300,559, filed on Nov. 20, 2002.
(60) Provisional application No. 60/333,109, filed on Nov. 20, 2001.

(51) Int. Cl. A61K 35/78; A61K 9/70

(52) U.S. Cl. 424/443; 424/778

(57) ABSTRACT

The present invention provides a method for preventing a respiratory infection in a mammal at risk thereof. The method includes contacting a live respiratory pathogen at risk of entering the respiratory tract of the mammal with a therapeutically effective amount of an essential oil. The live respiratory pathogen is inactivated upon contact with the essential oil. The source of the essential oil is a patch located in the vicinity of the nasal passageway of the mammal. The present invention also provides a kit that includes the adhesive patch, a mask for placing over the nasal passageway, packaging material, and optionally instructions for using the adhesive patch and mask.

### BACKGROUND OF THE INVENTION

Essential oils, also known as “essences” or “volatile oils,” are the highly odoriferous, liquid components obtained from plant tissue. Essential oils are usually captured by steam distillation, a process whose origins can be traced back to ancient Mesopotamia. Unlike ordinary vegetable oils, such as corn and olive, plant essences are highly volatile and will evaporate if left in the open air. The word “essential” is derived from quintessence, which the Oxford English Dictionary defines as “[a]n extract of a substance containing its principle in its most concentrated form”. In ancient philosophical or alchemical terms, quintessence was related to ether or the fifth element and was thought to be the spiritual aspect of matter. It is also interesting to mention that essential oils are sometimes called “ethereal oils”, a Germanic term which aptly describes their otherworldly nature; for if left in the open air they disappear without a trace, evaporating into the ether like a mist.

Essential oils may be found in different parts of the plant: in the petals (rose), leaves (eucalyptus), roots of grass (vetiver), bark (cinnamon), heartwood (sandalwood), citrus rind (lemon), seeds (caraway), rhizomes (valerian), bulbs (garlic), the aerial or top parts of the plant (marjoram) or resin (frankincense), and sometimes in more than one part of the plant. Lavender, for instance, yields oil from both the flowers and the leaves, while the orange tree produces three different smelling essences with varying medicinal properties; the heady bitter-sweet neroli (flowers), the similar though less refined scent of petit-grain (leaves) and the cheery orange (rind of the fruit).

Most essential oils consist of hundreds of components, such as terpenes and their oxygenated derivatives, alcohols, aldehydes, and carboxylic esters. For this reason a single oil can help a wide variety of disorders. Lavender, for instance, is endowed with antiseptic, antibacterial, antibiotic, antidepressant, analgesic, decongestant, and sedative properties. Moreover, due to their small molecular size, essential oils applied to the skin can readily enter into the bloodstream.

Quite apart from their medicinal properties, it is believed that just smelling an essential oil can uplift the spirits and make us feel better. This is because the sense of smell is an interrelated aspect of the limbic system, which is an area of the brain which is primarily concerned with emotion and memory. Indeed, this influence of aroma on the psyche has led some aromatherapists to practice what is now called “psycho-aromatherapy”, whereby oils are used solely as mood enhancing substances. By enabling a person to relax deeply, to let go of all their cares, even for just a while, it is potentially powerful enough to activate the body's own innate self-healing ability. Not only does aromatherapy alleviate stress and improve mood, it is a successful treatment for all manner of minor disorders for which doctors cannot always find a gentle solution, i.e., a solution free of the potentially harmful effects of drugs.

It is also believed that just smelling a particular scent can trigger a person to remember a specific event or occurrence. Many people who wish to recall or remember past events in their lives can utilize essential oils to accomplish this. This may also include people who are afflicted with mental, psychological, or neurological disorders (e.g., Alzheimer's Disease).

Many essential oils are superb skin care agents. They help to balance sebum (the skin's natural oil secretion), and to tone the complexion by supporting capillary function. Applied without massage, essential oils can heal skin problems such as athlete's foot, cold sores, ringworm and scabies. They can alleviate cold and flu symptoms and are also efficacious for problems such as coughs, tonsillitis, sore throats, sinusitis and acute bronchitis.

While each essential oil has its own unique properties, many also share some common therapeutic actions. All plant essences are antiseptics to a greater or lesser degree; good examples being eucalyptus, tea tree and thyme. Some oils are endowed with antiviral properties as well; garlic and tea tree being two of the most powerful. For obvious reasons, garlic essence is not usually employed in aromatherapy massage (though it has been known) but instead, is taken as a medicine in the form of garlic capsules. Many essences, notably rosemary and juniper, are also antirheumatic. When rubbed into the skin, they stimulate blood and lymphatic circulation and increase oxygen to the painful areas, which in turn aids the elimination of tissue wastes (uric and lactic acids, for example) which contribute to the pain of arthritic and rheumatic complaints.

Essential oils typically include a mixture of one or more terpenes, esters, aldehydes, ketones, alcohols, phenols, and/or oxides. These functional classes of compounds are responsible for the therapeutic properties of the essential oil. Specifically, common terpenes include limonene (an antiviral agent found in 90 percent of citrus oils), and pinene (an antiseptic found in high concentrations in pine and turpentine oils). Others, such as chamazulene and farnesol (found in chamomile essence), possess remarkable anti-inflammatory and bactericidal properties.

The most widespread group of esters found in plant essences include linalyl acetate (found in clary sage and lavender), and geranyl acetate (found in sweet marjoram). Esters are fungicidal and sedative, usually with a fruity odor. Aldehydes are found notably in lemon-scented essences, such as lemongrass and citronella. Aldehydes generally have a sedative, though uplifting, quality.

Certain ketones are known to be toxic, so this chemical group is regarded with a degree of caution. However, it is misleading to generalize about the toxicity of individual chemical components without knowing the exact ratio of the substance in relation to other chemicals in the whole oil. Certain essences, however, do contain appreciable quantities of toxic ketones, so should be avoided by lay people. Mugwort, tansy, wormwood and common sage contain the potentially risky thujone, while pennyroyal contains pulegone. Non-toxic ketones include jasmone, found in jasmine, and fenchone in sweet fennel. Ketones ease congestion and aid the flow of mucus, which is why plants and essences containing relatively large quantities of these substances are usually helpful for upper respiratory complaints.

Some of the most common alcohols include linalol (found in abundance in lavender), citronellol (rose, lemon, eucalyptus and geranium) and geraniol (geranium and palmarosa). These substances tend to have good antiseptic and antiviral properties and an uplifting quality. Phenols are bactericidal with a strong, stimulating effect on the central nervous system. Essential oils containing relatively large quantities of certain phenols are potentially irritant to skin and mucous membranes. Common caustic phenols include eugenol (found in clove essence), thymol (thyme) and carvacrol (oregano). However, anethole (from fennel) and estragole (tarragon) are not at all caustic. Oxides are found in a wide range of essences, especially those of a camphoraceous nature, such as rosemary, eucalyptus, tea tree and cajuput. Oxides tend to have an expectorant effect; for example, eucalyptol (eucalyptus).

Many essential oils are unstable at room temperature, especially over an extended period of time. This results in the necessity of essential oils being stored in cool dark places. Additionally, many essential oils are messy and inconvenient to use. This usually requires handlers of the essential oil to wash their hands after using the essential oil. It is also difficult for many handlers of essential oils to use the appropriate amount of essential oil, especially when the essential oil must be diluted immediately prior to use.

U.S. Pat Nos. 6,096,334; 6,096,333; 5,741,510; and 5,536,263 disclose adhesive patches that include a formulation that is partially embedded. The formulation can include methyl salicylate (oil of wintergreen), menthol, camphor, eucalyptus oil, or spearmint oil. Although the adhesive patches that include these compounds, alone or in combination with each other, have detectable odors that can illicit a response from a given individual, they are not disclosed or suggested to be able to kill airborne pathogens or respiratory tract pathogens.

U.S. Pat. No. 6,090,403 discloses an adhesive patch that includes a formulation. The formulation can include oil of wintergreen, menthol, thymol, camphor, oil of peppermint, eucalyptus oil, spirits of turpentine, ephedra, coltsfoot, or ginger. The formulation, however, is not disclosed or suggested to be partially embedded in the backing of the adhesive patch. Additionally, although the adhesive patches that include these compounds, alone or in combination with each other, are not disclosed or suggested to be able to kill airborne pathogens or respiratory tract pathogens.

U.S. Pat Nos. 4,675,009 and 4,307,717 disclose adhesive patches that include a formulation. The formulation can include cinnamon oil, fir needle oil, lemon oil, peppermint oil, Peruvian Balsam, or spearmint. The formulation, however, is not disclosed or suggested to be partially embedded in the backing of the adhesive patch. Additionally, although the adhesive patches that include these compounds, alone or in combination with each other, are not disclosed or suggested to be able to kill airborne pathogens or respiratory tract pathogens.

The above issued U.S. patents do not disclose that each of the essential oils disclosed therein (e.g., methyl salicylate (oil of wintergreen), menthol, camphor, eucalyptus oil, spearmint oil, thymol, oil of peppermint, spirits of turpentine, ephedra, coltsfoot, ginger, cinnamon oil, fir needle oil, lemon oil, and Peruvian Balsam; can be used in combination with one or more other essential oils. Specifically, the above issued U.S. patents do not disclose that the essential oils disclosed therein can be used in combination with one or more additional essential oils, and that some of the combinations result in at least one of the essential oils having a surprisingly synergistic effect (i.e., at least some of the essential oils, in combination, will have a greater therapeutic index than the combined therapeutic indexes of the individual essential oils).

Essential oils are known to have activity, e.g., against respiratory tract pathogens. See, e.g., Journal Antimicrobial Chemotherapy (2001) 47, 565-573; Journal Allergy Clinical Immunology, May 1996, 1133-1138; U.S. Pat. No. 5,322,689; U.S. Pat. No. 6,447,816; and Japanese Patent No. JP 5,306,217. These references, however, do not disclose devices that include such essential oils in known, discrete, safe, and effective amounts.

Accordingly, what is needed is a device that includes an essential oil. The device will include a known, discrete, safe, and effective amount of essential oil. The device will maintain the stability of the essential oil over an extended period of time. The device will also be convenient to use. Additionally, the device will allow for the prevention of a disease associated with an airborne pathogen or respiratory tract pathogen, utilizing an essential oil in a known, safe, efficient, and convenient manner.

### SUMMARY OF THE INVENTION

The present invention provides for the use of an adhesive patch in preventing and/or treating respiratory infections. The adhesive patch includes an essential oil in a known, discrete, safe, and effective amount. Specifically, the adhesive patch includes an essential oil in an amount permitted by the United States Food and Drug Administraion (FDA). The adhesive patch maintains the stability of the essential oil over an extended period of time. The adhesive patch is also convenient to use. Additionally, the adhesive patch allows for the prevention of other diseases associated with airborne pathogens, respiratory tract pathogens, or a combination thereof.

The present invention provides a method for preventing a respiratory infection in a mammal at risk thereof. The method includes contacting a live respiratory pathogen at risk of entering the respiratory tract of the mammal with a therapeutically effective amount of an essential oil, such that the live respiratory pathogen is inactivated upon contact with the essential oil, wherein the source of the essential oil is a patch located in the vicinity of the nasal passageway of the mammal.

The present invention also provides a method for preventing a respiratory viral infection in a mammal at risk thereof. The method includes contacting a live respiratory virus with a prophylactically effective amount of an essential oil, such that the live respiratory virus is inactivated upon contact with the essential oil, wherein the source of the essential oil is a patch located in the vicinity of the nasal passageway of the mammal.

The present invention also provides a method for preventing the transmission of a respiratory infection between mammals. The method includes contacting a live respiratory pathogen exiting the respiratory tract of a first mammal with a therapeutically effective amount of an essential oil, such that the live respiratory pathogen is inactivated upon contact with the essential oil, wherein the source of the essential oil is a patch located in the vicinity of the nasal passageway of the first mammal.

The present invention also provides a method for preventing the transmission of a respiratory infection between mammals. The method includes contacting a live respiratory pathogen at risk of entering the respiratory tract of a first mammal with a therapeutically effective amount of an essential oil, such that the live respiratory pathogen is inactivated upon contact with the essential oil, wherein the source of the essential oil is a patch located in the vicinity of the nasal passageway of the first mammal.

The present invention also provides a method for inhibiting a respiratory pathogen. The method includes contacting a live respiratory pathogen with a therapeutically effective amount of an essential oil, such that the live respiratory pathogen is inactivated upon contact with the essential oil, wherein the source of the essential oil is a patch located in the vicinity of the respiratory pathogen.

The present invention also provides a method for treating a respiratory infection in a mammal infected thereof or at risk thereof. The method includes contacting a live respiratory pathogen with a therapeutically effective amount of an essential oil, such that the live respiratory pathogen is inactivated upon contact with the essential oil, wherein the source of the essential oil is a patch located in the vicinity of the nasal passageway of the mammal.

The present invention also provides a kit that includes: (a) a patch that includes a flexible backing having a front side and a back side and a formulation positioned on at least a portion of the front side of the backing, in at least a portion of the front side of the backing, or on and in at least a portion of the front side of the backing, wherein the formulation that includes a therapeutically effective respiratory pathogen inhibiting amount of an essential oil; (b) a mask for placing over the nasal passageway of a mammal; and (c) packaging material.

The patch can include a flexible backing having a front side and a back side and a formulation positioned on at least a portion of the front side of the backing, in at least a portion of the front side of the backing, or on and in at least a portion of the front side of the backing; wherein the formulation includes the essential oil.

### BRIEF DESCRIPTION OF THE FIGURES

Embodiments of the invention may be best understood by referring to the following description and accompanying drawings which illustrate such embodiments. The numbering scheme for the Figures included herein are such that the leading number for a given reference number in a Figure is associated with the number of the Figure. For example, an adhesive patch 1 can be located in FIG. 1. However, reference numbers are the same for those elements that are the same across different Figures. In the drawings:

FIG. 1 illustrates the front side of an adhesive patch useful in the present invention.

FIG. 2 illustrates the back side of an adhesive patch useful in the present invention.

FIG. 3 illustrates the front side of an adhesive patch useful in the present invention, with a release liner attached to the patch.

FIG. 4 illustrates the back side of an adhesive patch useful in the present invention, with a release liner attached to the patch.

FIG. 5 illustrates the back side of an adhesive patch useful in the present invention, with a release liner attached to the patch and the patch is partially detached from the release liner.

FIG. 6 illustrates the back side of an adhesive patch useful in the present invention, with a release liner attached to the patch and the patch is partially detached from the release liner.

FIG. 7 illustrates a top view of a specific patch useful in the present invention.

FIG. 8 illustrates a top view of a specific patch useful in the present invention.

FIG. 9 illustrates a specific adhesive skin patch useful in the present invention, wherein the patch is in use.

FIG. 10 illustrates an enlarged cross-sectional view of a specific patch useful in the present invention.

### DETAILED DESCRIPTION OF THE INVENTION

The present invention provides for the use of an adhesive patch in preventing respiratory infections. The adhesive patch includes an essential oil in a known, discrete, safe, and effective amount. The adhesive patch maintains the stability of the essential oil over an extended period of time. The adhesive patch is also convenient to use. Additionally, the adhesive patch allows for the prevention of other disease associated with airborne pathogens, respiratory tract pathogens, or a combination thereof.

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The present invention provides a unique adhesive vehicle. The vehicle has pressure sensitive adhesive qualities due to its composition and viscoelastic nature. The adhesive is hydrophilic and therefore water can dissolve into or evaporate from the adhesive, depending on the conditions to which it is exposed. This water exchange capability implies that if the adhesive is on a suitably porous backing and is applied to the skin, it will not be occlusive as most drug delivery patches are. The occlusive nature of conventional drug delivery patches is thought to play an important role in enhancing drug absorption, but also often results in greater incidence of skin irritation. The relatively low occlusiveness of the present invention can be envisioned to be a special adhesive ointment or gel which is water-breathable, such as a water washable or water soluble ointment or gel.

The present invention provides an ointment or gel on a backing. The ointment or gel includes an essential oil in a known, discrete, safe, and effective amount. The adhesive patch maintains the stability of the essential oil over an extended period of time. The backing is pliable and/or stretchable. Since the backing can be porous and/or vapor permeable, many consumers typically refer to the device as a “patch,” a “skin patch,” or an “adhesive skin patch.” As such, the device (i.e., the ointment or gel on the backing) will herein be referred to as a patch, a skin patch, an adhesive skin patch and/or as an antiviral inhalation patch. It is appreciated that those skilled in the art understand that the term “patch” is used to refer to the device and is not otherwise limiting in any manner.

It is appreciated that those of skill in the art understand that the terms used herein, unless expressly stated otherwise, include the singular as well as the plural. For example, the term “essential oil” 15 includes the singular (i.e., one essential oil) as well as the plural (i.e., two or more essential oils).

As used herein, “holdout” refers to the physical properties of a backing, relating to the ability of a specific class of gels or ointments to penetrate, cross- link, wet, and/or cure within the matrix of the backing. A specific class of gels or ointments may or may not be able to penetrate a given backing. Upon penetration of a gel or ointment into a backing, the gel or ointment will cross-link, wet, or cure in the backing. As such, the holdout properties are the ability of the gel or ointment to affect the degree of penetration, cross-linking, wetting, and/or curing within the matrix of the backing. Those backings with superior holdout properties will typically prevent, decrease, or lessen the likelihood of the ointment or gel from wetting the backing; will typically increase the likelihood of the ointment or gel to cross-link within the matrix of the backing; will typically increase the likelihood of the ointment or gel to cure within the matrix of the backing; and/or will typically prevent, decrease, or increase the likelihood of the ointment or gel to partially penetrate the matrix of the backing.

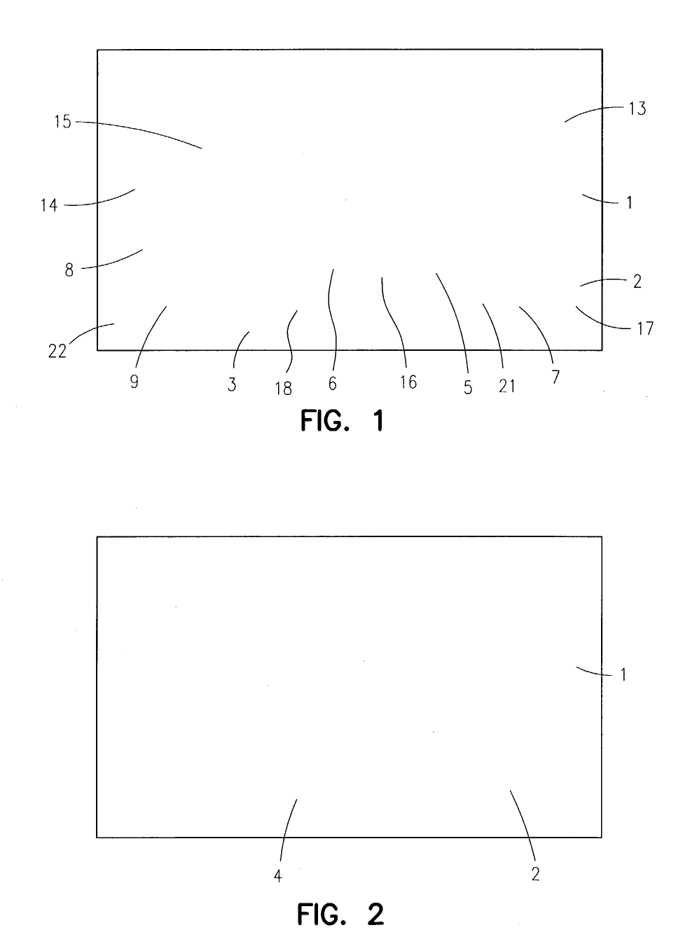

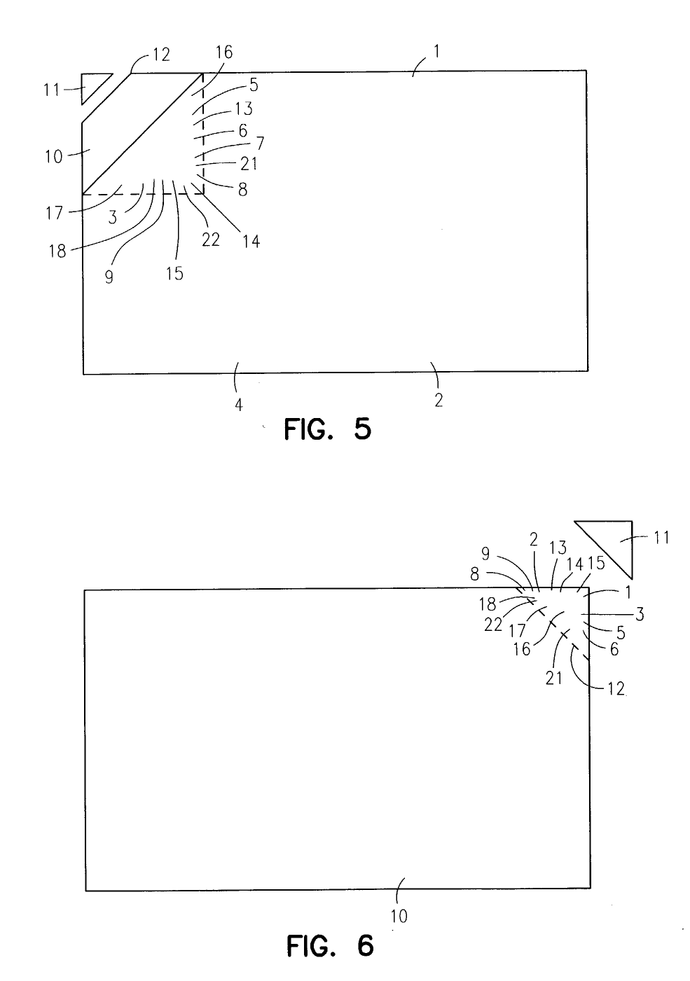

Referring to FIGS. 1-10, an adhesive patch 1 of the present invention is provided. The adhesive patch 1 includes a formulation 5 located on at least a portion of the front side 3 of the backing 2, in at least a portion of the front side 3 of the backing 2, or on and in at least a portion of the front side 3 of the backing 2. Preferably, the formulation 5 is partially embedded in at least a portion of the front side 3 of the backing 2. In addition to being located in at least a portion of the front side 3 of the backing 2, the formulation 5 is located on a portion of the surface of front side 3 of the backing 2. Preferably, the formulation 5 is located on the entire surface of the front side 3 of the backing 2.

Backing

The backing 2 is defined by a front side 3 (the side exposed to the skin during use) and a back side 4 (the side exposed to the environment during use). The backing 2 should be nonirritating to human skin. The backing 2 is a self-supporting sheet of water soluble or water insoluble, polymeric or natural material that provides strength and integrity for the formulation 5. The backing 2 of the adhesive patch 1 can be vapor permeable. The backing 2 can also be porous, since porosity provides openings for receiving the formulation 5 and it helps to assure that the adhesive skin patch 1 is vapor permeable. Specifically, the backing 2 can retain the formulation 5 while allowing moisture from the skin to pass. The backing 2 can have any suitable thickness, provided the suitable thickness allows for a flexible, bendable, pliable, vapor permeable, and/or a stretchable sheet of water insoluble porous material. Specifically, the thickness of the backing 2 can be about 0.001 mm to about 5.0 mm, about 0.001 mm to about 3.0 mm, or about 0.025 mm to about 1.25 mm.

The backing 2 can be manufactured from any suitable material, provided the suitable material can form a flexible, bendable, pliable, and/or stretchable backing 2. The backing 2 includes a flexible porous sheet of water soluble or water insoluble material that provides support for the adhesive skin patch 1. The backing 2 can include water soluble or water insoluble polymeric fibers, a porous film, or any other kind of matrix with spaces within the matrix. A specific backing 2 is a lightweight, porous, pliable strip composed of a nonwoven fabric of polymeric or natural fibers such as polyester, cotton or cellulose fibers bonded together with a sizing resin. The backing 2 can be woven or nonwoven. Preferably, the backing 2 includes nonwoven fabric.

Specifically, the backing 2 can include polyester fibers, polyurethane fibers, polyolefin fibers, polyamide fibers, natural fibers, cotton fibers, copolyester fibers, cellulose acetate fibers, polyceilulose fibers, or any mixture thereof. Additional stable, water insoluble flexible sheet materials and methods for manufacturing the suitable backings 2 are disclosed, e.g., in U.S. Pat. No. 4,675,009; U.S. Pat. No. 5,536,263; U.S. Pat. No. 4,696,854; U.S. Pat. No. 5,741,510, and references cited therein, and are suitable as backings 2 according to the present invention. The infusion of the formulation 5 into the backing 2 can be accomplished, e.g., with the use of a continuous process mixer, as disclosed, e.g., in U.S. Pat. No. 5,536,263, and references cited therein; or as discussed herein.

Alternatively, the backing 2 can be a non-woven backing 2 that is treated by coating: the front side 3 of the backing adhesive patch 1, the back side 4 of the backing 2, or both the front side 3 and back side 4 of the backing 2; with a silicone-containing compound, a fluorocarbon solution, or a combination thereof. Suitable silicone-containing compounds include, e.g., polydimethyl siloxanes, dialkylsiloxanes, dimethylsiloxo vinyl alkenes, dialkylsiloxo vinyl alkenes, dimethylsiloxo acrylates, dialkylsiloxo acrylates, vinyl terminated polydimethylsiloxane, and vinyl terminated polydialkylsiloxane. The exemplary silicone-containing compounds are commercially available from, e.g., Goldschmidt Chemical Corp. (Essen, Germany); GE Silicones (Waterford, N.Y.); Wacker Silicone Corp. (Adrian, Mich.); and Dow Corning Corp. (Midland, Mich.).

The backing 2 can be manufactured from a suitable non-woven fabric that is commercially available from, e.g., Freudenberg Faservliesstoffe KG (Weinham, Germany); Sontara Technologies (division of DuPont Corporation) (Old Hickory, Tenn.); Lystil S. A. (Brignoud Cedex, France); Dexter Nonwovens (Windsor Locks, Conn.); and Chicopee (New Brusnwick, N.J.). Other commercial vendors that supply suitable non-woven fabrics can be found at the Technical Textile website (http://www.technical-textiles.net/technical-textiles-index/orgL.htm).

It has surprisingly been discovered that the use of a treated backing, such as a fluorocarbon treated non-woven backing, typically increases the yield of an adhesive patch. The use of a backing material that has been treated with a sizing agent allows for the effective control of the rate of penetration, such that the gel or ointment has solidified after it has begun to penetrate the backing, but before it has passed completely through the backing. In addition, the use of a backing material that has been treated with a sizing agent allows for the effective control of the depth to which the ointment or gel will easily penetrate before solidifying. It has surprisingly been discovered that increasing the control of the rate at which the ointment or gel penetrates the backing typically improves the overall yield of the production process by reducing the amount of material which must be discarded because the back side of the backing has become too tacky for either processing or for consumer acceptance.

At least a portion of the backing 2 can be treated with a sizing agent 8 such that the portion of the backing 2 that is treated with the sizing agent 8 has a surface energy of about 20 dynes/cm² to about 65 dynes/cm². Specifically, the portion of the backing 2 that is treated with the sizing agent 8 can have a surface energy of about 27 dynes/cm² to about 56 dynes/cm². The sizing agent 8 lowers the surface energy of the portion of the backing 2 that is treated with the sizing agent 8. Any suitable sizing agent 8 can be employed, provided the portion of the backing 2 that is treated with the sizing agent 8 has a surface energy of about 20 dynes/cm² to about 65 dynes/cm². Suitable sizing agents 8 include, e.g., fluorocarbon solutions, silicone-containing compounds, and combinations thereof. Specifically, the backing 2 can be a non-woven backing 2 that is treated with a fluorocarbon. For example, the fluorocarbon treated backing 2 can be, e.g., Vilmed M1585 W/HY, Vilmed M1585H/HY, Vilmed M1586 W/HY, Vilmed M1586 H/HY, Vilmed M1570, Vilmed M1573 F, Vilmed M1573 FH, Vilmed M1577 F, Vilmed M1578 F, or Vilmed M1578 FH; which are all commercially available from Freudenberg Faservliesstoffe KG (Weinham, Germany). Alternatively, the silicone treated backing 2 can be a non-woven backing 2 that is coated with one or more silicone-containing compounds, e.g., a polydimethyl siloxane, a dialkylsiloxane, a dimethylsiloxo vinyl alkene, a dialkylsiloxo vinyl aikenes, a dimethylsiloxo acrylate, a dialkylsiloxo acrylate, a vinyl terminated polydimethylsiloxane, and a vinyl terminated polydialkylsiloxane.

At least a portion of the backing 2 can be treated with the sizing agent 8. The portion of the backing 2 that is treated with the sizing agent 8 can be that portion of the backing 2 that can typically include the formulation 5. The entire surface of the front side 3 of the backing 2 can be treated with the sizing agent 8 or a portion of the surface of the front side 3 of the backing 2 can be treated with the sizing agent 8. Preferably, the entire surface of the front side 3 of the backing 2 can be treated with the sizing agent 8. In addition to the surface of the front side 3 of the backing 2 being treated with the sizing agent 8, the sizing agent 8 can penetrate at least a portion of the underlying surface (e.g., one-tenth to about nine-tenths the thickness, or about one-fourth to about nine-tenths the thickness) of the backing 2. Specifically, the sizing agent 8 can penetrate the entire underlying surface of the backing 2.

Suitable fluorocarbon treated backings 2 include, e.g., Vilmed M1585 W/HY, Vilmed M1585H/HY, Vilmed M1586 W/HY, Vilmed M1586 H/HY, Vilmed M1570, Vilmed M1573 F, Vilmed M1573 FH, Vilmed M1577 F, Vilmed M1578 F, and Vilmed M1578 FH; which are all commercially available from Freudenberg Faservliesstoffe KG (Weinham, Germany).

As shown in FIGS. 1-6 and 9-10, the backing 2 includes a front side 3 and a back side 4. The adhesive skin patch 1 includes a formulation 5 located in at least a portion of the front side 3 of the backing 2, on at least a portion of the front side 3 of the backing 2, or on and in at least a portion of the front side 3 of the backing 2. As such, the formulation 5 can be located on the entire surface of the front side 3 of the backing 2 or the formulation 5 can be located on a portion of the surface of the front side 3 of the backing 2.

Preferably, the formulation 5 can be located on the entire surface of the front side 3 of the backing 2. In addition to being located on the surface of the front side 3 of the backing 2, the formulation 5 can be located in at least a portion of the underlying surface of the front side 3 of the backing 2 (i.e., the formulation 5 can be partially embedded into the backing 2).

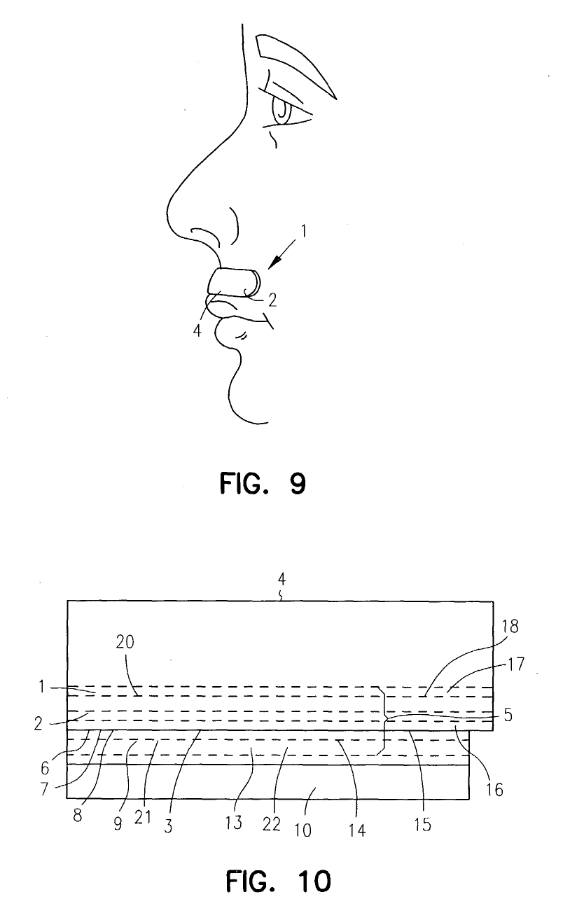

As shown in FIG. 9, the formulation 5 can penetrate a substantial portion of the front side 3 of the backing 2, as disclosed, e.g., in U.S. Pat. No. 5,536,263, and references cited therein. For example, the formulation 5 can penetrate about one-tenth to about nine-tenths the thickness of the backing 2, or about one-fourth to about nine-tenths the thickness of the backing 2. As such, the formulation 5 can be partially embedded into the backing 2. Preferably, the formulation 5 can be located on the entire front side 3 of the backing 2 and partially in the front side 3 of the backing 2 (i.e., the formulation 5 is partially embedded into the backing 2).

Alternatively, a portion of the front side 3 of the backing 2 can include the formulation 5 and other portions of the front side 3 of the backing 2 can include any combination of the pressure sensitive adhesive 14 and solvent 13. For example, a central circular portion of the front side 3 of the backing 2 can include the formulation 5 while the remaining portions of the front side 3 of the backing 2 include only the pressure sensitive adhesive 14. The formulation 5, when partially embedded into the front side 3 of the backing 2, imparts strength and structure into the adhesive patch 1. For example, when the formulation 5 is partially embedded into the backing 2, the likelihood that the adhesive patch 1 will tear apart when separated from the release liner 10 or when removed from the skin after use, is minimized.

When the adhesive skin patch 1 is placed upon the skin of a patient (e.g., human), the formulation 5 can be in continuous contact with the skin surface of the patient.

Preferably, the adhesive skin patch 1, upon contact with skin, will allow the skin to breathe. More preferably, the adhesive skin patch 1, upon prolonged contact with skin, will hold in place the formulation 5 and will permit the skin to breathe over prolonged periods of time typically experienced with the use of the patch, e.g., up to about 24 hours, up to about 12 hours, up to about 8 hours, or up to about 6 hours.

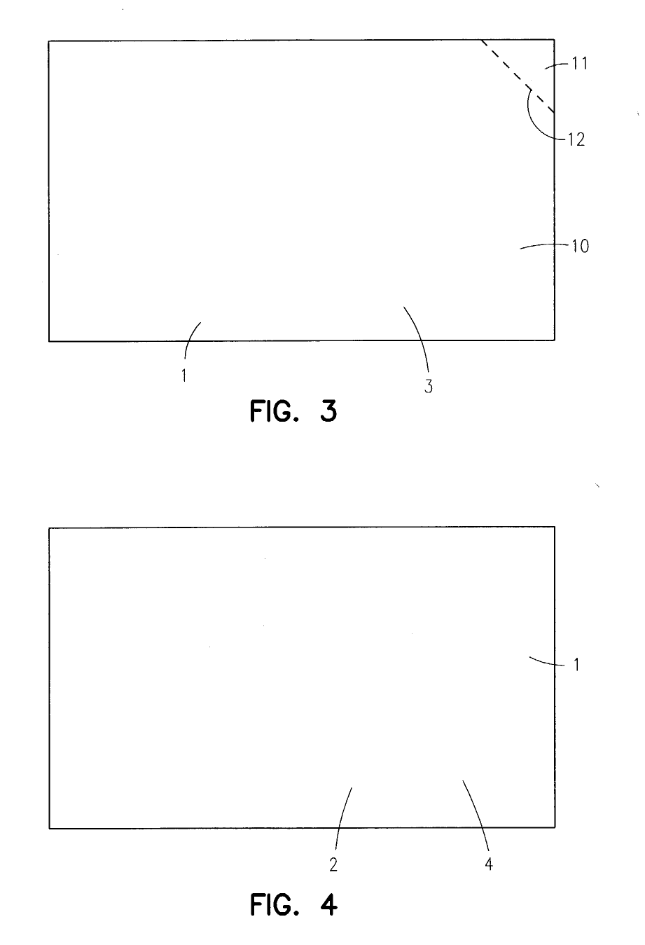

As shown in FIGS. 3-6 and 9, the adhesive skin patch 1 can be reversibly attached to a release liner 10. The release liner 10 helps to maintain the adhesiveness of the adhesive skin patch 1 prior to use, such as during manufacturing, packaging, shipping, and/or storage. Any suitable release liner 10 can be employed for use in the present invention. Suitable release liners 10 are readily known to those of skill in the art. See, e.g., U.S. Pat. No. 4,675,009; U.S. Pat. No. 5,536,263; U.S. Pat. No. 4,696,854; U.S. Pat. No. 5,741,510, and references cited therein for further descriptions of release liners 10 useful in the present invention. The release liner 10 can include a perforation 12 that allows the tab section 11 of the release liner 10 to be removed (see, FIGS. 3, 5, and 6). Removal of the tab section 11 of the release liner 10 allows the adhesive skin patch 1 to be removed from the release liner 10 with relative ease.

Essential Oil

As used herein, an “essential oil” 15 refers to a highly odoriferous, volatile liquid component obtained from plant tissue. Essential oils 15 typically include a mixture of one or more terpenes, esters, aldehydes, ketones, alcohols, phenols, and/or oxides. These functional classes of compounds are responsible for the therapeutic properties and distinct fragrance of the essential oil.

In one specific embodiment of the present invention, the essential oil 15 is not: methyl salicylate, menthol, camphor, eucalyptus oil, spearmint oil, or a combination thereof. In another specific embodiment of the present invention, the formulation 5 of the present invention can include any one or more of methyl salicylate, menthol, camphor, eucalyptus oil, or spearmint oil; provided another essential oil 15 is included in the formulation 5.

In one embodiment of the present invention, the formulation 5 can include methyl salicylate, menthol, camphor, eucalyptus oil, spearmint oil, or a combination thereof. In such an embodiment, the formulation 5 will also include one or more additional essential oils 15.

In one specific embodiment of the present invention, the essential oil 15 is not: oil of wintergreen, thymol, oil of peppermint, spirits of turpentine, ephedra, coltsfoot, ginger, cinnamon oil, fir needle oil, lemon oil, Peruvian Balsam, or a combination thereof. In another specific embodiment of the present invention, the formulation 5 of the present invention can include any one or more of oil of wintergreen, thymol, oil of peppermint, spirits of turpentine, ephedra, coltsfoot, ginger, cinnamon oil, fir needle oil, lemon oil, Peruvian Balsam, or a combination thereof; provided another essential oil 15 is included in the formulation 5.

In one embodiment of the present invention, the formulation 5 can include oil of wintergreen, thymol, oil of peppermint, spirits of turpentine, ephedra, coltsfoot, ginger, cinnamon oil, fir needle oil, lemon oil, Peruvian Balsam, or a combination thereof. In such an embodiment, the formulation 5 will also include one or more additional essential oils 15.

The essential oil 15 can be manufactured (i.e., synthesized or partially synthesized). Alternatively, the essential oil 15 can be obtained from a plant or plant component (e.g., plant tissue). Suitable plant or plant components include, e.g., a herb, flower, fruit, seed, bark, stem, root, needle, bulb, berry, rhizome, rootstock, leaf, or a combination thereof.

Any suitable essential oil 15 can be employed provided:

(1) the essential oil 15 has the desired therapeutic and/or prophylactic properties (e.g., the essential oil 15 effectively kills or inactivates an airborne pathogen, a respiratory tract pathogen, or a combination thereof); and

(2) the essential oil 15 remains stable in the formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. The specific essential oil 15 will preferably be non-toxic to mammals (e.g., humans) and will be suitable for medicinal use (e.g., topically and/or via inhalation). The specific essential oil 15 will also preferably comply with any controlling or governing body of law, e.g., FDA regulations.

Suitable specific essential oils 15 include, e.g., one or more of the following: ajowan, sweet almond oil, allspice, aloe vera oil, ammi visnaga (khella), amyris, angelica root, angelica seed, anise, anise seed, star anise, apricot kernel oil, absolute arnica, avocado oil, unrefined avocado oil, Copaiba balsam, balsam Peru genuine, balsam Peru oil, balsam peru liquid resin, balsam tolu, sweet french basil, basil, basil ct. methyl chavicol, lemon ct. citral basil, sweet ct. linalool basil, bay laurel, bay leaf, bay rum, bay leaf West Indies, bees wax, unrefined bees wax, benzoin absolute, benzoin resinoid, bergamot, mint bergamot, Italian bergamot oil, free bergaptene bergamot, birch, sweet birch, borage oil, boronia, butter, buchu leaf, cajeput, calamus, calendula oil, infused calendula oil, camellia oil, cannabis, caraway, caraway seed, cardamom, absolute carnation, carrot seed, high carotol carrot seed, carrot seed oil, cassia, cassis bud (black currant), castor oil, catnip, oil of catnip, cedarleaf, western red cedarleaf, cedarwood, Atlas cedarwood, Himalayan cedarwood, Virginia cedarwood, celery seed, chamomile, blue chamomile, German chamomile, Moroccan chamomile, Moroccan wild chamomile, Roman chamomile, champaca, cilantro, true cinnamon bark, cinnamon bark, cinnamon leaf, cinnamon cassia, cistus, citronella, Java citronella, ciste oil, artificial civet, clary sage, high sclareol clary sage, clementine, Italian clementine peel oil, clove, clove bud, clove leaf, cocoa, cocoa butter, unrefined cocoa butter, coconut oil, refined coconut oil, cognac, combava petitgrain, coriander, green coriander, cornmint, costus oil, cumin, cypress, davana oil, dill, dill weed, elemi, erigeron (fleabane), eucalyptus citriodora, eucalyptus globulus, lemon eucalyptus, fennel, sweet fennel, fenugreek, fir, Canada fir needle, Siberia fir needle, white fir needle, frankincense, India frankincense, Oman frankincense, galbanum oil, garlic, genet, geranium, geranium leaf, geranium rose, Bourbon geranium, Egyptian geranium, ginger, Cochin extra ginger, ginsing, Siberian ginsing, Korean ginsing, grapefruit, pink grapefruit, white grapefruit, grapeseed oil, hazelnut oil, helichrysum, helichrysum immortelle, Mad. helichrysum, Balkan helichrysum, Corsica helichrysum, France helichrysum, hemp oil, absolute honeysuckle, hyssop, hyssop decumbens, absolute immortelle, fragrant aster inula, Jamaican gold, unrefined Jamaican gold, jasmine, absolute jasmine, grandiflorum jasmine, sambac jasmine, jojoba oil, helio-carrot in jojoba, melissa in jojoba, absolute jonquille, juniper berry, Siberia juniper berry, Croatia juniper berry, lanolin, unrefined anhydrous lanolin, lantana camara, laurel nobilis, lavandin, abrialis lavandin, grosso lavandin, lavender, Oregon lavender, Bulgarian lavender, Russian lavender, high-altitude lavendar, wild-crafted lavender, lavendin, organic lavindin, lemon, lemongrass, lime, distilled lime, expressed lime, litsea, litsea cubeba, blue, pink and white lotus, macadamia oil, mace, green mandarin, red mandarin, yellow mandarin, manuka, absolute marigold, marigold flower, marjoram, Spanish marjoram, sweet marjoram (true), massoia bark, melissa, codistilled melissa, “rectified” melissa, true melissa, absolute mimosa, mimosa, monarda, mugwort, musk seed, myrrh, myrtle, absolute narcissus, neroli (orange blossom), niaouli, nutmeg, extra nutmeg, oakmoss, absolute oak moss, olibanum, absolute opopanax, bitter orange, blood orange, sweet orange, wild West Indian orange, oregano, orris root, concrete orris, osmanthus, palm oil, refined palm oil, palmarosa, paprika, parsley seed, patchouli, Indian patchouli oil, Indonesian patchouli oil, peanut, peanut oil, pecan oil, pennyroyal, pepper, black pepper, super black pepper, peppermint, India peppermint, USA baby mint peppermint, pet perfume, petitgrain (orange leaves), white pine, pine needle, evening primrose, ravensara anisata, true ravensara, ravensare, ravintsara, redberry, rosalina, rose, rose geranium, rose otto, Bulgarian rose, English rose, Turkish rose, rosehip seed oil, rosemary, rosemary anti-oxidant extract powder, rosemary verbenone, Morocco rosemary, Spain rosemary, rosewood, rosewood oil, rue, sage, white sage, sage dalmatian, sage officinalis, sage triloba, sandalwood, seabuckthorn berry, sesame oil, sesame seed oil, shea butter, unrefined shea butter, spikenard, green spikenard, spruce, St. John's wort, styrax resin, tagetes, tangerine, Dancy tangerine, tarragon, tea tree, Australia tea tree, thuja (cedar leaf), thyme, red thyme, thyme ct. linalool, thyme vulgaris, wild thyme, red thyme, mixed tocopherols, tolu balsam resin, absolute tuberose, tuberose, tumeric, valerian, vanilla, pure vanilla extract, vanilla bean, absolute vanilla bourbon, vegetable glycerin, absolute verbena, vetiver, violete leaves, vitex, organic Haiti vetiver, absolute violet leaf, walnut oil, wintergreen, natural wintergreen, wormwood, yarrow, ylang ylang, ylang ylang I, ylang ylang II, ylang ylang III, ylang ylang compound, ylang ylang complete, and ylang ylang extra.

Specifically, suitable exemplary essential oils 15 include, e.g., angelica root, anise, basil (e.g., sweet French basil), bay leaf, benzoin absolute, bergamot, birch, carrot seed, cedarwood, chamomile (e.g., German chamomile, Moroccan chamomile, or Roman chamomile), cinnamon leaf, cinnamon cassia, cistus, citronella, clary sage, clove bud, cypress, eucalyptus globulus, eucalyptus citriodora, everlasting (helicrysum), fennel, fir, frankincense, geranium, ginger, grapefruit, helichrysum, hyssop, juniper berry, lavender, lavendin, lemon, lemongrass, lime, marjoram, myrrh, myrtle, neroli, niaouli, nutmeg, sweet orange, oregano, patchouli, pennyroyal, peppermint, petitgrain, pepper, pine needle, ravensare, rose geranium, rosemary (e.g., Spanish rosemary), rosewood, sage, sandalwood, spikenard, spruce, tangerine, tarragon, tea tree, thyme, vanilla, vetiver, ylang ylang, or a combination thereof.

Other suitable essential oils 15 that can be employed in the adhesive skin patch 1 of the present invention are are disclosed in the following websites: www.essential-essences.com; www.fragrancefactory.com; www.essentialoil.com; www.essentialoils.org; www.halcyon.com; and www.essential-oil.org; which are all incorporated by reference herein.

The essential oil 15 can be present in any appropriate and suitable amount, provided:

(1) the amount of essential oil 15 has the desired therapeutic and/or prophylactic properties (e.g., the essential oil 15 effectively kills or inactivates an airborne pathogen, respiratory tract pathogen, or a combination thereof); and

(2) the amount of essential oil 15 remains stable in the formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. The specific amount of essential oil 15 will preferably be non-toxic to mammals (e.g., humans) and will be suitable for medicinal use (e.g., topically and/or via inhalation). The specific amount of essential oil 15 will also preferably comply with any controlling or governing body of law, e.g., FDA regulations.

Typically, the amount of essential oil 15 present in the formulation 5 will depend upon the specific compound or compounds employed as the essential oil 15. Specifically, the essential oil 15 can be present in about 0.01 wt. % to about 99.9 wt. % of the formulation 5. More specifically, the essential oil 15 can be present up to about 50 wt. % of the formulation 5, up to about 25 wt. % of the formulation 5, up to about 20 wt. % of the formulation 5, up to about 10 wt. % of the formulation 5, or up to about 5 wt. % of the formulation 5.

In one embodiment of the present invention, angelica root, anise, basil (e.g., sweet French basil), bay leaf, benzoin absolute, bergamot, birch, carrot seed, cedarwood, chamomile (e.g., German chamomile, Moroccan chamomile, or Roman chamomile), cinnamon leaf, cinnamon cassia, cistus, citronella, clary sage, clove bud, cypress, eucalyptus globulus, eucalyptus citriodora, everlasting (helicrysum), fennel, fir, frankincense, geranium, ginger, grapefruit, helichrysum, hyssop, juniper berry, lavender, lavendin, lemon, lemongrass, lime, marjoram, myrrh, myrtle, neroli, niaouli, nutmeg, sweet orange, oregano, patchouli, pennyroyal, peppermint, petitgrain, pepper, pine needle, ravensare, rose geranium, rosemary (e.g., Spanish rosemary), rosewood, sage, sandalwood, spikenard, spruce, tangerine, tarragon, tea tree, thyme, vanilla, vetiver, ylang ylang, or a combination thereof, or any combination thereof, can be present up to about 20 wt. % of the formulation 5, up to about 10 wt. % of the formulation 5, or up to about 5 wt. % of the formulation 5.

The adhesive skin patch 1 includes an essential oil 15 located in at least a portion of the front side 3 of the backing 2, on at least a portion of the front side 3 of the backing 2, or on and in at least a portion of the front side 3 of the backing 2. As such, the essential oil 15 can be located on the entire surface of the front side 3 of the backing 2 or the essential oil 15 can be located on a portion of the surface of the front side 3 of the backing 2. Preferably, the essential oil 15 can be located on the entire surface of the front side 3 of the backing 2.

In addition to being located on the surface of the front side 3 of the backing 2, the essential oil 15 can be located in at least a portion of the underlying surface of the front side 3 of the backing 2 (i.e., the essential oil 15 can be partially embedded into the backing 2). As shown in FIG. 9, the essential oil 15 can penetrate a substantial portion of the front side 3 of the backing 2, as disclosed, e.g., in U.S. Pat. No. 5,536,263, and references cited therein. For example, the essential oil 15 can penetrate about one-tenth to about nine-tenths the thickness of the backing 2, or about one-fourth to about nine-tenths the thickness of the backing 2. As such, the essential oil 15 can be partially embedded into the backing 2.

Preferably, the essential oil 15 can be located on the entire front side 3 of the backing 2 and partially in the front side 3 of the backing 2 (i.e., the essential oil 15 is partially embedded into the backing 2). Alternatively, a portion of the front side 3 of the backing 2 can include the essential oil 15 and other portions of the front side 3 of the backing 2 can include the pressure sensitive adhesive. For example, a central circular portion of the front side 3 of the backing 2 can include the essential oil 15 while the remaining portions of the front side 3 of the backing 2 include only the pressure sensitive adhesive 14. When the adhesive skin patch 1 is placed upon the skin of a patient (e.g., human), the essential oil 15 can be in continuous contact with the skin surface of the patient.

As used herein, “treating” or “treat” includes (i) preventing a pathologic condition (e.g., respiratory infection) from occurring (e.g. prophylaxis); (ii) inhibiting the pathologic condition (e.g., respiratory infection) or arresting its development; and (iii) relieving the pathologic condition (e.g., respiratory infection), or symptoms related to the same.

As used herein, “mammal” refers to a class of vertebrate animals of more than 15,000 species, including humans, distinguished by self-regulating body temperature, hair, and in the females, milk-producing mammae. Specifically, mammal can refer to a human.

Plant Tissue

The essential oil 15 can be derived from plant tissue.

As used herein, “plant tissue” refers to the tissue of any organism of the plant kingdom, as opposed to one of the animal kingdom or of the kingdoms of Fungi, Protista, or Monera. The plant tissue can be any portion or portions of the plant (e.g., bark, roots, leaves, flowers, needles, bulbs, berries, rhizomes, rootstocks, stems, and seeds), as well as the entire plant. The tissues of a plant (“plant tissue”) generally fall into three main categories: dermal tissue, ground tissue, and vascular tissue. Dermal tissue refers to the “skin” layer of all plant organs and is responsible for environmental interaction (light passage, gas exchange, pathogen recognition and protection, color display, etc.). Dermal tissue is composed of epidermal cells, closely packed cells that secrete a waxy cuticle that aids in the prevention of water loss. Ground tissue lies between dermal tissue and vascular tissue. The ground tissue comprises the bulk of the primary plant body. Parenchyma, collenchyma, and sclerenchyma cells are common in the ground tissue. In roots, the ground tissue may store sugars or starches to fuel the spring sap flow; in leaves, the ground tissue is the layer responsible for photosynthesis (the mesophyll). Vascular tissue transports food, water, hormones and minerals within the plant. Vascular tissue includes xylem, phloem, parenchyma, and cambium cells.

As used herein, “bark” refers to the dry, dead outer covering of woody branches, stems and roots of plants that is very distinct and separable from the wood itself. It includes all tissue outside the cambium (growth layer between bark and wood).

As used here the terms “leaf” or “leaves” refer to those parts of a plant which grow along the sides of branches or stems or at the bases of plants. Most are green and contain chlorophyll, though they vary in their shapes and sizes. Leaves are the part of the plant that ordinarily performs photosynthesis (the process that converts sunlight and carbon dioxide into energy).

As used herein, “needle” generally refers to a narrow stiff leaf, such as those of conifers (e.g., pine trees).

As used herein, “root” refers to the part of a plant, normally underground, that absorbs nutrients and anchors the plant into the ground.

As used herein, “bulb” refers to a spheroidal body growing from a plant either above or below the ground (usually below), which is usually a bud, consisting of a cluster of partially developed leaves, and producing, as it grows, a stem above, and roots below, (e.g., the onion or tulip bulb). A true bulb is a complete package containing next year's plant (flower) already forming inside. The contents of the bulb are often enclosed in protective, fleshy scales, which are held together by a small basal plate. The scales are modified leaves that contain enough nutrients to sustain the plant through dormancy and early growth. They may be loose and open like those of a lily, or tightly closed like those of a hyacinth. In many bulbs, a paper-thin tunic protects the scales (lilies don't have a tunic). Roots will grow from the bulb's basal plate.

As used herein, “berry” refers to any small fruit that is pulpy or succulent throughout, having seeds loosely imbedded in the pulp, such as the currant, grape, or blueberry. Berry can be further defined as an indehiscent fruit derived from a single ovary and having the whole wall fleshy, such as the grape or tomato. Furthermore, berries come in various structures including simple, such as grape; blueberry, cranberry, or aggregate, such as blackberry; raspberry, strawberry mulberry.

As used herein, “rhizome” refers to a horizontal, usually underground stem that often sends out roots and shoots from its nodes (also called rootstalk or rootstock).

As used herein, “rootstock” refers to a robust plant that provides the root system in grafting, also known as a stock. Scions and buds are grafted and budded to a rootstock or stock. Rootstock also refers to the elongated and often thick rhizomes of certain perennial herbaceous plants such as the Iris, Aspidistra and Solomon's Seal.

As used herein, “stem” refers to the main (usually aerial) axis (sometimes referred to as the trunk or stalk) of a tree, shrub, or plant. “Stem” also refers to the part of the plant that supports the leaves, flowers or fruits of a plant, such as the peduncle of a fruit or the pedicel of a flower.

As used herein, “seed” refers to a ripened ovule, consisting of an embryo with one or more integuments, or coverings, such as an apple seed, a currant seed, dill seed, or kola nut seed. By germination, most seeds produce a new plant. “Seed” also refers to any small seedlike fruit, though it may consist of a pericarp, or even a calyx, as well as the seed proper, such as a parsnip seed or thistle seed. The seed proper has an outer and an inner coat, and within these the kernel or nucleus. The kernel is either the embryo alone, or the embryo enclosed in the albumen, which is the material for the nourishment of the developing embryo. The scar on a seed, left where the stem parted from it, is called the hilum, and the closed orifice of the ovule, the micropyle.

Solvent

When present, the solvent 13 can act as a carrier for, and preferably can dissolve, the essential oil 15 and/or the pressure sensitive adhesive 14. Any suitable solvent 13 can be employed, provided the solvent 13 effectively dissolves the essential oil 15 and/or the pressure sensitive adhesive 14 and the solvent 13 remains stable in the formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1.

The solvent 13 can include one or more organic compounds, one or more inorganic compounds, or mixtures thereof. Preferably, the solvent 13 will include one or more organic compounds, e.g., esters, terpenes, alcohols, ketones, aldehydes, fatty acids, partially or fully esterified fatty acids, wherein the structures are cyclic, non cylcic (e.g., alkyl), alicyclic (i.e., a bridged ring compound), or aromatic, as well as organic compounds having combinations of these functional groups. Suitable exemplary solvents 13 are disclosed, e.g., in Aldrich Handbook of Fine Chemicals, 2000-2001 (Milwaukee, Wis.).

Preferably, the solvent 13 includes a polyhydric alcohol, water, or a combination thereof. The polyhydric alcohol can be erythritol, propylene glycol, ethylene glycol, triethylene glycol, or a combination thereof. Erythritol is commercially available from Cragill (Minnetonka, Minn.). Additional suitable solvents 13 include, e.g., glycerin; triacetin; diethylene glycol methyl ether; diethylene glycol methyl ether acetate; 1,3-propane ricinoleate; PEG-6 caprylic/capric glycerides; caprylic/capric triglycerides; propyleneglycol dicaprylate/dicaprate; glycerol monostearate; glycerol monocaprylate; glycerol monolaurate; neopentyl alcohol; 1-hexadecanol; hydroxypropyl beta-cyclodextrin; vitamin E; vitamin E acetate; deoxycholic acid; taurodeoxycholic acid; 3-[(3-cholamidopropyl) dimethylammonio]-1-propane-sulfonate; BigCHAP; cholic acid; cholesterol NF; propylene carbonate; lecithin; a medicinally acceptable salt thereof; or a combination thereof.

When present, the solvent 13 can be employed in any suitable amount, provided the amount of solvent 13 is effective to dissolve the essential oil 15 and/or the pressure sensitive pressure sensitive adhesive 14 and the effective amount of solvent 13 remains stable in the formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. Specifically, the solvent 13 can be present in about 1.0 wt % to about 70.0 wt. %; in about 3.0 wt % to about 50.0 wt. %; or in about 5 wt. % to about 30 wt. % of the formulation 5. Typically, the amount of solvent 13 will depend on the compound or compounds employed as the solvent 13. For example, a polyhydric alcohol can be present up to about 70 wt. % of the formulation 5; or in about 20.0 wt. % to about 60.0 wt. % of the formulation 5; and water can be present in about 2.0 wt. % to about 50.0 wt. % of the formulation 5.

When present, the solvent 13 can be located in at least a portion of the front side 3 of the backing 2, on at least a portion of the front side 3 of the backing 2, or on and in at least a portion of the front side 3 of the backing 2. As such, the solvent 13 can be located on the entire surface of the front side 3 of the backing 2 or the solvent 13 can be located on a portion of the surface of the front side 3 of the backing 2. Preferably, the solvent 13 can be located on the entire surface of the front side 3 of the backing 2. In addition to being located on the surface of the front side 3 of the backing 2, the solvent 13 can be located in at least a portion of the underlying surface of the front side 3 of the backing 2 (i.e., the solvent 13 can be partially embedded into the backing 2).

As shown in FIG. 9, the solvent 13 can penetrate a substantial portion of the front side 3 of the backing 2, as disclosed, e.g., in U.S. Pat. No. 5,536,263, and references cited therein. For example, the solvent 13 can penetrate about one-tenth to about nine-tenths the thickness of the backing 2, or about one-fourth to about nine-tenths the thickness of the backing 2. As such, the solvent 13 can be partially embedded into the backing 2. Preferably, the solvent 13 can be located on the entire front side 3 of the backing 2 and partially in the front side 3 of the backing 2 (i.e., the solvent 13 is partially embedded into the backing 2). Alternatively, a portion of the front side 3 of the backing 2 can include the solvent 13 and other portions of the front side 3 of the backing 2 can include any combination of the pressure sensitive adhesive 14 and essential oil 15. When the adhesive skin patch 1 is placed upon the skin of a patient (e.g., human), the solvent 13 can be in continuous contact with the skin surface of the patient.

Pressure Sensitive Adhesive

The formulation 5 can further include a pressure sensitive adhesive. Any suitable pressure sensitive adhesive 14 can be employed, provided the pressure sensitive adhesive 14 provides the requisite adhesiveness to the adhesive skin patch 1 and the pressure sensitive adhesive 14 remains stable in the formulation 5.

Preferably, the stability is over a prolonged period of time, e.g., up to about 3 years, up to about 1 year, or up to about 6 months, typically experienced in the manufacturing, packaging, shipping, and/or storage of the adhesive skin patch 1. It is appreciated that the suitable pressure sensitive adhesives 14 are known to those skilled in the art. Suitable pressure sensitive adhesives 14 are disclosed, e.g., in U.S. Pat. No. 4,675,009; U.S. Pat. No. 5,536,263; U.S. Pat. No. 4,696,854; U.S. Pat. No. 5,741,510, and references cited therein. Preferably the pressure sensitive adhesive 14 is an acrylic ester copolymer.

Any suitable amount of pressure sensitive adhesive 14 can be employed, provided the amount of pressure sensitive adhesive 14 effectively provides the requisite adhesiveness to the adhesive skin patch 1 and the effective amount of the pressure sensitive adhesive 14 remains stable in the formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. The formulation 5 can include a pressure sensitive adhesive 14 in about 0.1 wt. % to about 50 wt. %, in about 0.5 wt. % to about 10.0 wt. %, or in about 1.0 wt. % to about 15.0 wt. % of the formulation 5.

Typically, the suitable amount of pressure sensitive adhesive 14 will depend upon the specific pressure sensitive adhesive 14 employed. For example, the pressure sensitive adhesive 14 can include one or more acrylic ester copolymers. Each of the one or more acrylic ester copolymers can be present up to about 20.0 wt. % of the formulation 5. Specifically, each of the acrylic ester copolymers can be present up to about 40.0 wt. % of the formulation 5, or up to about 30.0 wt. % of the formulation 5. More specifically, all of the one or more acrylic ester copolymers, when combined, can be present in about 3.0 wt. % to about 40.0 wt. % of the formulation 5, or in about 5.0 wt. % to about 30.0 wt. % of the formulation 5. As such, the total amount of acrylic ester copolymers can be about 3.0 wt. % to about 40.0 wt. % of the formulation 5, or about 5.0 wt. % to about 30.0 wt. % of the formulation 5.

Alternatively, the pressure sensitive adhesive 14 can include a hot melt pressure sensitive adhesive 14 or solvent based pressure sensitive adhesive 14 (e.g., polyacrylate, polyisobutylene, and polybutene), rubber, silicone based pressure sensitive adhesives 14 (e.g., polydimethylsiloxane and resin mixtures), polystyrene-polybutadiene-polystyrene, polystyrene-polyisoprene-polystyrene, polystyrene-poly(ethylene-butylene)-polystyrene block polymers, or any combination thereof. In addition, the adhesive 14 can include a resin emulsion adhesive, wherein the resin emulsion adhesive can include vinyl acetate resin, acrylic ester copolymer, vinyl acetate/diocyl maleate copolymer, acrylic copolymer, or any combination thereof.

Other suitable pressure sensitive adhesives 14 are disclosed, e.g., in U.S. Pat. No. 4,675,009; U.S. Pat. No. 5,536,263; U.S. Pat. No. 4,696,854; U.S. Pat. No. 5,741,510, and references cited therein.

The pressure sensitive adhesive 14 can be located in at least a portion of the front side 3 of the backing 2, on at least a portion of the front side 3 of the backing 2, or on and in at least a portion of the front side 3 of the backing 2. As such, the pressure sensitive adhesive 14 can be located on the entire surface of the front side 3 of the backing 2 or the pressure sensitive adhesive 14 can be located on a portion of the surface of the front side 3 of the backing 2. Preferably, the pressure sensitive adhesive 14 can be located on the entire surface of the front side 3 of the backing 2.

In addition to being located on the surface of the front side 3 of the backing 2, the pressure sensitive adhesive 14 can be located in at least a portion of the underlying surface of the front side 3 of the backing 2 (i.e., the pressure sensitive adhesive 14 can be partially embedded into the backing 2). As shown in FIG. 9, the pressure sensitive adhesive 14 can penetrate a substantial portion of the front side 3 of the backing 2, as disclosed, e.g., in U.S. Pat. No. 5,536,263, and references cited therein. For example, the pressure sensitive adhesive 14 can penetrate about one-tenth to about nine-tenths the thickness of the backing 2, or about one-fourth to about nine-tenths the thickness of the backing 2. As such, the pressure sensitive adhesive 14 can be partially embedded into the backing 2.

Preferably, the pressure sensitive adhesive 14 can be located on the entire front side 3 of the backing 2 and partially in the front side 3 of the backing 2 (i.e., the pressure sensitive adhesive 14 is partially embedded into the backing 2). Alternatively, a portion of the front side 3 of the backing 2 can include the pressure sensitive adhesive 14 and other portions of the front side 3 of the backing 2 can include the essential oil 15. The pressure sensitive adhesive 14 , being partially embedded into the front side 3 of the backing 2, imparts strength and structure into the adhesive patch 1.

For example, when the pressure sensitive adhesive 14 is partially embedded into the backing 2, the likelihood that the adhesive patch 1 will tear apart when separated from the release liner 10 or when removed from the skin after use, is minimized. When the adhesive skin patch 1 is placed upon the skin of a patient (e.g., human), the pressure sensitive adhesives 14 can be in continuous contact with the skin surface of the patient.

Emulsifier

The formulation 5 or pressure sensitive adhesive 14 can optionally include a compound that emulsifies the formulation 5 or the pressure sensitive adhesive 14. One suitable compound that effectively emulsifies the formulation 5 or the pressure sensitive adhesive 14 is pectin. The emulsifier (e.g., pectin) can be present in any suitable amount, provided the suitable amount is effective to emulsify the formulation 5 or the pressure sensitive adhesive 14 and the emulsifier remains stable in the formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. Specifically, the emulsifier (e.g., pectin) can be present up to about 30.0 wt. % of the formulation 5, in about 1.0 wt. % to about 20.0 wt. % of the formulation 5, or in about 2.0 wt. % to about 10.0 wt. % of the formulation 5.

Polymer

The pressure sensitive adhesive 14 can optionally include one or more polymers 9. The polymer 9 provides structure and strength to the pressure sensitive adhesive 14 or provides structure and strength to the formulation 5. Any suitable polymer 9 can be employed, provided the polymer 9 provides structure and strength to the pressure sensitive adhesive 14 or provides structure and strength to the formulation 5, and the polymer 9 remains stable in the formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1.

Suitable polymers 9 include natural polymers and synthetic polymers. Specifically, the polymer 9 can include, e.g., karaya, a polyacrylamide, xanthum gum, guar gum, a hydrophilic polymer, a hydrocolloidal polymer, starch, a starch derivative, vinyl acetate copolymer, polyvinyl pyrrolidone, polyethylene oxide, algin, a derivative of algin, a polyacrylate, gelatin, polymaleic acid, polyacrylic acid, polymaleic anhydride, a polyurethane, a polyurea, gum acacia, locust bean gum, modified guar gum, maltodextrin, carboxymethyl cellulose, carboxypropyl cellulose, polyvinyl alcohol, poly AMPS, or a combination thereof. Other suitable polymers 9 are disclosed, e.g., in U.S. Pat. No. 4,675,009; U.S. Pat. No. 5,536,263; U.S. Pat. No. 4,696,854; U.S. Pat. No. 5,741,510, and references cited therein. Preferably, the polymer 9 can include polyacrylamide, karaya, or a combination thereof.

Any suitable amount of polymer 9 can be employed, provided the amount of polymer 9 effectively provides structure and strength to the pressure sensitive adhesive 14 or to the formulation 5, and the effective amount of polymer 9 remains stable in the formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. Typically, the suitable amount of polymer 9 will depend upon the specific polymer 9 employed. Specifically, karaya can be employed as the polymer 9 up to about 60 wt. % of the formulation 5, in about 5.0 wt. % to about 45 wt. % of the formulation 5, or in about 8.0 wt. % to about 40 wt. % of the formulation 5; polyacrylamide can be employed as the polymer 9 up to about 40 wt. % of the formulation 5, in about 5.0 wt. % to about 35 wt. % of the formulation 5, or in about 8.0 wt. % to about 30 wt. % of the formulation 5; or both karaya and polyacrylamide can be employed as the polymer 9, wherein karaya is present in about 5.0 wt. % to about 35 wt. % of the formulation 5 and polyacrylamide is present in about 5.0 wt. % to about 30 wt. % of the formulation 5.

Humectant

The formulation 5 can optionally include one or more humectants 17 to provide a moistening effect to the pressure sensitive adhesive 14. The humectant 17 can optionally hydrate the polymer 9. Any suitable humectant 17 can be employed, provided the humectant 17 effectively provides a moistening effect to the pressure sensitive adhesive 14 and the humectant 17 remains stable in the formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. One suitable humectant 17 is glycerin. Other suitable humectants 17 include polyhydric alcohols such as ethylene glycol, propylene glycol, triethylene glycol, tetraethylene glycol, sorbitol, and combinations thereof.

Any suitable amount of humectant 17 can be employed, provided the amount of humectant 17 effectively provides a moistening effect to the pressure sensitive adhesive 14 and the amount of humectant 17 effectively remains stable in the formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. Typically, the suitable amount of humectant 17 will depend upon the specific humectant 17 employed and the specific polymer 9 employed. For example, karaya, polyacrylamide, or a combination thereof can be employed as the polymer 9 and glycerin can be employed as the humectant 17, wherein the glycerin is present in about 25.0 wt. % to about 70.0 wt. % or in about 40.0 wt. % to about 55.0 wt. % of the formulation 5.

Filler

The formulation 5 can optionally include one or more fillers 6. Any suitable filler 6 can be employed. Suitable fillers 6 include malto dextrin, dextrin, 70% sorbitol water, modified starches, depolymerized starches, and methylcellulose. As used herein, “malto dextrin” is a dextrose equivalent, wherein dextrose is D-glucose. Malto dextrin is commercially available as Amaizo Lodex 5 from American Maize-Products (Hammond, IN). Any suitable amount of filler 6 can be employed in the formulation 5. The suitable amount of filler 6 can depend in part upon the specific filler present in the formulation 5. For example, malto dextrin can be present up to about 20.0 wt. % of the formulation 5, or in about 1.0 wt. % to about 10.0 wt. % of the formulation 5.

Skin Protectant or Skin Conditioner

The formulation 5 can optionally include a skin protectant 18 (i.e., topical moisturizer or skin conditioner). Any suitable skin protectant 18 can be employed, provided the skin is effectively protected or moisturized and the skin protectant remains stable in the formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. Additionally, it is preferable that the skin conditioner is medicinally acceptable for topical use in humans.

Suitable topical moisturizers 18 include, e.g. calamine, aloe, lanolin, glycerin, Vitamin E, Vitamin E acetate, farnesol, glycyrrhetinic acid, aluminum hydroxide gel, cocoa butter, propylene glycol, ethylene glycol, triethylene glycol, hard fat, kaolin, mineral oil, petrolatum, topical starch, white petroleum, cod liver oil, shark liver oil, zinc oxide, or any combination thereof. Additional suitable topical moisturizers 18 are disclosed, e.g., in U.S. Pat Nos. 6,096,334; 6,096,033; 5,741,510; 5,536,263; 4,675,009; 4,307,717; 4,274,420; 5,976,565; 5,536,263; and references cited therein.

As used herein, “aluminum hydroxide gel” refers to a suspension containing aluminum oxide (Al203), mainly in the form of a hydroxide. It is typically obtained by drying the product of interaction in aqueous solution of an aluminum salt with ammonium or sodium carbonate.

As used herein, “cocoa butter” refers to a fatty substance in cocoa beans; a thick oily solid obtained from cocoa beans and used in making chocolate, cosmetics, and suntan oil. Also known as threobroma oil, it lubricates and softens the skin.

As used herein, “topical starch” refers to cornstarch.

As used herein, “kaolin” refers to aluminum silicate; powdered and freed from gritty particles by elutriation. Kaolin refers to the name of the locality in China where the substance is found in abundance.

As used herein, “white petroleum” refers to a purified mixture of hydrocarbons obtained from petroleum. A bleached version of yellow soft paraffin, it is used as an emollient and as a base for ointments. It is odorless when rubbed into the skin and not readily absorbed.

As used herein, “mineral oil” refers to the heavy liquid petrolatum; liquid paraffin or petroleum; a mixture of liquid hydrocarbons obtained from petroleum, and is typically used as a vehicle in medicinal preparations.

As used herein, “petrolatum” refers to petroleum jelly; a yellow soft paraffin; a yellowish mixture of the softer members of the paraffin or methane series of hydrocarbons, obtained from petroleum as an intermediate product in the distillation; typically used as a soothing application to burns and abrasions of the skin, and as a base for ointments.

As used herein, “cod liver oil” refers to the partially destearinated fixed oil extracted from the fresh livers of Gadus morrhuae and other species of the family Gadidae, containing Vitamins A and D.

As used herein, “shark liver oil” refers to the oil extracted from the livers of sharks, mainly of the species Hypoprion brevirostris; a rich source of Vitamins A and D.

As used herein, “zinc oxide” refers to ZnO, which is typically used as a protective ointment.

As used herein, “calamine” is a pink powder of zinc oxide and a skin protectant containing about 98% zinc oxide and about 0.5% ferric oxide; “aloe” is the dried latex of leaves of Curaco Aloe (Aloe barbadenis Miller, Aloe vera Linne) or Cape Aloe (Aloe ferox Miller and hybrids), of the family Liliacaea. Aloe is commercially available as Aloe Vera Gel from Terry Laboratories (Melbourne, Fla.). Aloe Vera Gel is commercially available as Aloe Vera Gel 40× (20.0 wt. % solution in water), Aloe Vera Gel 1× (0.5 wt. % solution in water), Aloe Vera Gel 10× (5.0 wt. % solution in water), or solid Aloe Vera. The solid Aloe Vera can be dissolved in a carrier, such as water, to the desired concentration. In addition, the commercially available forms of Aloe Vera are optionally available as decolorized Aloe Vera.

As used herein, “Vitamin E” is 3,4-dihydro-2,5,7,8-tetramethyl-2-(4,8,12-trimethyltridecyl)-2H-1-benzopyran-6-ol; “Vitamin E acetate” is 3,4-dihydro-2,5,7,8-tetramethyl-2-(4,8,12-trimethyltridecyl)-2H-1-benzopyran-6-ol acetate; “lanolin” is the fat-like secretion of the sebaceous glands of sheep (i.e., complex mixture of esters and polyesters of 33 high molecular weight alcohols and 36 fatty acids) which is deposited onto the wool fibers; “farnesol” is 3,7,11-trimethyl-2,6,10-dodecatrien-1-ol. Farnesol is commercially available from American Radiolabeled Chemicals (ARC) (St. Louis, Mo.), and “glycyrrhetinic acid” is a pentacyclic triterpenoid derivative of the beta-amyrin type and is shown below:

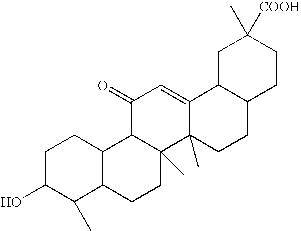

Any suitable amount of skin protectant 18 can be employed, provided the suitable amount of skin protectant 18 effectively protects or moisturizes the skin and the effective amount of skin protectant 18 remains stable in the formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. Additionally, it is preferable that the amount of skin conditioner employed is medicinally acceptable for topical use in humans.

Specifically, the skin protectant 18 can be present up to about 20.0 wt. %, up to 10.0 wt. %, up to 5.0 wt. %, or up to 2.0 wt. % of the formulation 5. The suitable and effective amount of skin protectant 18 will depend in part upon the specific skin protectant 18 present in the formulation 5. For example, Aloe Vera Gel, 10× can be present up to about 20.0 wt. % of the formulation 5, up to about 10.0 wt. % of the formulation 5, up to about 5.0 wt. % of the formulation 5, or up to about 1.0 wt. % of the formulation 5. In addition, Vitamin E acetate can be present up to about 10.0 wt. % of the formulation 5, up to about 5.0 wt. % of the formulation 5, up to about 3.0 wt. % of the formulation 5, up to about 2.0 wt. % of the formulation 5, or up to about 1.0 wt. % of the formulation 5. Preferably, the skin conditioner will be present in an amount that is consistent with any State or Federal regulations.

The skin protectant 18 can be located in at least a portion of the front side 3 of the backing 2, on at least a portion of the front side 3 of the backing 2, or on and in at least a portion of the front side 3 of the backing 2. As such, the skin protectant 18 can be located on the entire surface of the front side 3 of the backing 2 or the skin protectant 18 can be located on a portion of the surface of the front side 3 of the backing 2. Preferably, the skin protectant 18 can be located on the entire surface of the front side 3 of the backing 2. In addition to being located on the surface of the front side 3 of the backing 2, the skin protectant 18 can be located in at least a portion of the underlying surface of the front side 3 of the backing 2 (i.e., the skin protectant 18 can be partially embedded into the backing 2). As shown in FIG. 9, the skin protectant 18 can penetrate a substantial portion of the front side 3 of the backing 2, as disclosed, e.g., in U.S. Pat. No. 5,536,263, and references cited therein. For example, the skin protectant 18 can penetrate about one-tenth to about nine-tenths the thickness of the backing 2, or about one-fourth to about nine-tenths the thickness of the backing 2. As such, the skin protectant 18 can be partially embedded into the backing 2. Preferably, the skin protectant 18 can be located on the entire front side 3 of the backing 2 and partially in the front side 3 of the backing 2 (i.e., the skin protectant 18 is partially embedded into the backing 2). Alternatively, a portion of the front side 3 of the backing 2 can include the skin protectant 18 and other portions of the front side 3 of the backing 2 can include any combination of the solvent 13, pressure sensitive adhesive 14, and essential oil 15. When the adhesive skin patch 1 is placed upon the skin of a patient (e.g., human), the skin protectant 18 can be in continuous contact with the skin surface of the patient.

Preservative

The formulation 5 can optionally include a preservative 7 that is useful for preventing bacterial growth, mold growth, fermentation, and/or decomposition. As used herein, “preservative” refers to any substance which prevents bacterial growth, mold growth, fermentation, and/or decomposition. Concise Chemical and Technical Dictionary, 4th enlarged edition, Chemical Publishing Co., Inc., NY, N.Y. p. 939 (1986). Any suitable preservative 7 can be employed, provided the preservative 7 effectively prevents bacterial growth, mold growth, fermentation, and/or decomposition; and the preservative 7 remains stable in the formulation 5. Preferably, the stability is over a prolonged period of time, e.g., up to about 2 years, up to about 1 year, or up to about 6 months, typically experienced in the manufacturing, packaging, shipping, and/or storage of the adhesive skin patch 1.

Suitable preservatives 7 include, e.g., quat-15, parabens, dichlorobenzyl alcohol, ethylene diamine tetreacetic acid, formaldehyde, gum benzoin, imidazolidinyl urea, phenyl-mercuric acetate, poly aminopropyl biguanide, proply gallate, sorbic acid, cresol, chloroacetamide sodium benzoate, chloromethyl-methylisothiazolinone, chloromethyl-methylisothiazolon, chloromethyl-methylisothiazolinone benzalkonium chloride, an octylisothiazolinone benzimidazol-compound, chloromethyl-methylisothiazolinone octylisothiazolinone, o-phenylphenol benzisothiazolinone, o-phenylphenol benzisothiazolinone, benzisothiazolinone, an aliphatic amine of 2-thiopyridineoxide, benzoic acid, editic acid, phenolic acid, benzyl alcohol, isopropyl alcohol, benzenethonium chloride, bronopol, cetrimide, chiorohexidine, chlorobutanol, chlorocresol, phenol, phenoxyethanol, phenyl ethyl alcohol, phenylmercuric acetate, phenylmercuric borate, phenylmercuric nitrate, potassium sorbate, proplyene glycol, sodium benzoate, sodium propionate, thimerosol, and medicinally acceptable salts thereof. Preferably, the preservative is quat-15, which is commercially available from Dow Chemical (Midland Michigan); methyl paraben; ascorbic acid; or a combination thereof.

The preservative 7 can be employed in any suitable amount provided the amount of preservative 7 effectively prevents bacterial growth, mold growth, fermentation, and/or decomposition and the effective amount of preservative 7 remains stable in the formulation 5. Preferably, the stability is over a prolonged period of time, e.g., up to about 3 years, up to about 1 year, or up to about 6 months, typically experienced in the manufacturing, packaging, shipping, and/or storage of the adhesive skin patch 1. The preservative 7 can be present up to about 99.9 wt. % of the formulation 5, up to about 20.0 wt. % of the formulation 5, up to 5.0 wt. % of the formulation 5, or up to 1.5 wt. % of the formulation 5. The amount of preservative 7 present in the formulation 5 will typically depend upon the specific compound or compounds employed as the preservative 7. For example, quat-15 can be employed in about 0.01 wt. % to about 1.5 wt. % of the formulation 5, in about 0.05 wt. % to about 0.15 wt. % of the formulation 5, or in about 0.08 wt. % to about 0.12 wt. % of the formulation 5.

Antiviral Agent

As used herein, an “antiviral agent” is a compound or combination of compounds that weakens or abolishes the action of a virus. Stedman's Medical Dictionary, 25th Ed., illustrated, Williams & Wilkins, Baltimore, Md., p. 101 (1990). Any suitable antiviral agent 15 can be employed, provided the antiviral agent 15 effectively treats a viral infection and the antiviral agent 15 remains stable in the therapeutic formulation 5. Preferably, the stability is over a prolonged period of time, e.g., up to about 2 years, up to about 1 year, or up to about 6 months, typically experienced in the manufacturing, packaging, shipping, and/or storage of the patch 1.

Suitable antiviral agents are disclosed, e.g., in Physician's Desk Reference (PDR), Medical Economics Company (Montvale, N.J.), (53rd Ed.), 1999; Mayo Medical Center Formulary, Unabridged Version, Mayo Clinic (Rochester, Minn.), Jan. 1998; Merck Index, An Encyclopedia of Chemicals, Drugs, and Biologicals, (11th Ed.), Merck & Co., Inc. (Rahway, N.J.), 1989; and references cited therein. Suitable antiviral agents 15 include, e.g., zinc, lysine, foscarnet, 3-deoxythmidin-2-ene, dideoxycytosine, dideoxyinosine, lamivudine, azidothymidine, indinavir, ritonavir, saquinavir, acyclovir, idoxuridine, ribavirin, vidarabine, amantidine, rinantidine, viracea2, cytovene, famciclovir, valaciclovir, penciclovir, nonoxynol-9, pharmaceutically acceptable salts thereof, and combinations thereof. Additional suitable antiviral agents 15 include, e.g., a hypochloride, a hypochloride generating compound, a peroxide, a peroxide generating compound, an organic halide, an organic halide generating compound, or a combination thereof.

In a specific embodiment of the present invention, the antiviral agent 15 can include lysine hydrochloride.

The antiviral agent 15 can be present in any appropriate and suitable amount, provided the amount of antiviral agent 15 is effective to treat a viral infection and the amount of antiviral agent 15 remains stable in the therapeutic formulation 5 over a prolonged period of time. Typically, the antiviral agent 15 can be present in about 0.01 wt. % to about 99.9 wt. % of the therapeutic formulation 5. The amount of antiviral agent 15 present in the therapeutic formulation 5 will typically depend upon the specific compound or compounds employed as the antiviral agent 15. For example, lysine hydrochloride can be present up to about 99.9 wt. % of the therapeutic formulation 5, up to about 50 wt. % of the therapeutic formulation 5, or up to 20 wt. % of the therapeutic formulation 5. Preferably, the amount of antiviral agent 15 employed in the therapeutic formulation 5 will comply with FDA regulations.

Specifically, lysine hydrochloride can be present up to about 10.0 wt. % of the therapeutic formulation 5. Preferably, lysine hydrochloride can be present up to about 4.0 wt. % of the therapeutic formulation 5. More preferably, lysine hydrochloride can be present in about 0.01 wt. % to about 10.0 wt. % or in about 0.1 wt. % to about 4.0 wt. % of the therapeutic formulation 5.

The antiviral agent 15 can preferably be located on and in any portion of the therapeutic formulation 5, which is located on the front side 3 of the backing 2. Preferably, the antiviral agent 15 can be located on and in the entire portion of the therapeutic formulation 5. When the adhesive skin patch 1 is placed upon the skin of a patient (e.g., human), the antiviral agent 15 can be in continuous contact with the skin surface of the patient.

Complexing Agent

In one embodiment of the present invention, the formulation 5 can include an essential oil 15 that is not soluble and/or stable either without a solvent or with the specific solvent 13, in the amount employed. The use of a complexing agent can be employed to modulate (i.e., regulate) the solubility, stability, and/or the volatility of the essential oil 15 in the formulation 5. Any suitable complexing agent can be employed, provided the completing agent effectively solubilizes and/or stabilizes the essential oil 15 and the complexing agent remains stable in the formulation 5 over a prolonged period of time. Preferably, the stability is over a prolonged period of time, e.g., up to about 3 years, up to about 1 year, or up to about 6 months, typically experienced in the manufacturing, packaging, shipping, and/or storage of the adhesive skin patch 1. In addition, any suitable amount of complexing agent can be employed, provided the amount of complexing agent effectively solubilizes and/or stabilizes the essential oil 15 and the amount of complexing agent remains stable in the formulation 5 over a prolonged period of time.

Suitable specific complexing agents include, e.g., cyclodextrins. As used herein, a “cyclodextrin” refers to a non-reducing cyclic oligosaccharide with at least 6 anhydroglucose units linked by alpha 1,4 bonds to form a ring. Cyclodextrins are typically produced by the action of the enzyme cyclodextrin glucosyltransferase [CGT-ase] on starch. The most common cyclodextrins include alpha, beta, and gamma cyclodextrins, which have six, seven, or eight, respectively, anhydroglucose units in the ring structure. All of the hydroxyl groups in cyclodextrins are oriented to the outside of the ring while the glucosidic oxygen and two rings of the non-exchangeable hydrogen atoms are directed towards the interior of the cavity. This combination gives cyclodextrins a hydrophobic inner cavity and a hydrophilic exterior. See, e.g., the Cerestar website (http://www.cerestar.com); the Betadexcyclodextrin website (http://www.betadexcyclodextrin.com); and M. L. Bender and M. Komiyama, Cyclodextrin Chemistry, Springer, Berlin, 1978.

Cyclodextrins are enzymatically-modified starches formed by the action of the enzyme cyclodextrin glucosyltransferase on starch. They are doughnut-shaped molecules, which can interact with organic molecules to form complexes. It is also possible for some organic molecules and some inorganic salts to associate with the hydroxyl groups of the cyclodextrin. Three cyclodextrins are typically formed, alpha, beta, and gamma cyclodextrin, which contain six, seven, or eight glucose molecules in the ring, respectively. The electron-dense glycosidic oxygen atoms are oriented inward and line the cavity. The hydroxyl groups are directed toward the outside of the ring. These hydrophilic groups interact with the water to give the cyclodextrins their aqueous solubility properties. The hydrogen and glycosidic oxygen atoms lining the cavity give the cyclodextrin molecule its hydrophobic character and its ability to interact with organic molecules to form complexes. Because of the free rotation of the C-6 carbon, this end of the cyclodextrin cavity is narrower than the end with the C-2 and C-3 hydroxyls.

Derivatives of cyclodextrin can be obtained, e.g., by replacing one or more hydroxyl groups with a suitable radical (i.e., pendant group). Suitable pendant groups include, e.g., sulfinyl; sulfonyl; phosphate; (C1-C12)alkyl optionally substituted with one or more (e.g., 1, 2, 3, or 4) hydroxy, carboxy, carbonyl, acyl, oxy, oxo; or a combination thereof. Suitable specific pendant groups include methyl, ethyl, hydroxypropyl, carboxymethyl, sulfate, phosphate, and an acrylate. For example, the specific pendant group can include (C1-C12)alkoxy optionally substituted with one or more hydroxy.

Specific suitable derivatives of cyclodextrin include, e.g., alpha-cyclodextrin sulfate, beta-cyclodextrin sulfate, gamma-cyclodextrin sulfate, alpha-hydroxypropyl cyclodextrin, beta-hydroxypropyl cyclodextrin, gamma-hydroxypropyl cyclodextrin, aipha-cyclodextrin phosphate, beta-cyclodextrin phosphate, and gamma-cyclodextrin phosphate.

Cyclodextrins are starches that have been specially modified by the action of an enzyme to make a water-soluble ring-shaped molecule, capable of holding another, oil-like organic substance in its ‘cavity’. Because of this unique property, cyclodextrins can be used to carry all kinds of active ingredients (e.g., drugs, fragrances, flavors, and vitamins) in a wide variety of formulations. Increased stability, water solubility, and controlled release are among the many application benefits. Specifically, cyclodextrins have the benefit of encapsulating a substance, thereby providing protection for the substance. This results in increased shelf-life and a reduced loss of degradation or decomposition. Cyclodextrins are themselves soluble in water, and can greatly increase the solubility of highly water insoluble substances. In addition, cyclodextrins can be used to control the release of a substance.

Suitable cyclodextrins include alpha cyclodextrins, beta cyclodextrins, and gamma cyclodextrins. Specifically, the cyclodextrin can be hydroxylpropyl beta cyclodextrin, hydroxylproplyl alpha cyclodextrin, or a combination thereof. In addition, the cyclodextrin can optionally be branched.

Suitable cyclodextrins, and derivatives thereof, can be found, e.g., at U.S. Pat. No. 5,376,641; U.S. Pat. No. 5,229,370; U.S. Pat. No. 4,383,992; the Cerestar website (http://www.cerestar.com); the Betadexcyclodextrin website (http://www.betadexcyclodextrin.com); French et al., Archives in Biochem. and Biophysics, Volume III, (1965) 153-150; the carbomer website (http://www.carbomer.com) and references cited therein.

In one embodiment of the present invention, an adhesive skin patch 1 is provided in which the formulation 5 does not include an essential oil 15. In such an embodiment, the adhesive skin patch 1 can be manufactured, shipped, and stored without an essential oil 15 and an essential oil 15 can be introduced to the adhesive skin patch 1 at a later time.

The formulation 5 can preferably remain stable over the period of time typically experienced with the manufacturing, packaging, shipping, and/or storage of the adhesive skin patch 1, e.g., up to about a month, up to about a year, up to about two years, or up to about 3 years. The stability of the essential oil 15, for example, is due in part to the formulation 5 including the essential oil 15 in an adhesive formulation. The adhesive formulation is preferably a hydrogel that holds the essential oil 15 in an available form while maintaining the necessary stability, pressure sensitive adhesion and effectiveness over a prolonged period of time, e.g., up to about a month, up to about a year, up to about two years, or up to about 3 years.

The adhesive skin patch 1 can have any suitable size and shape. In addition, the adhesive skin patch 1 can be cut, as desired, to provide an adhesive skin patch 1 of a desired size and shape. The adhesive skin patch 1 can be cut with any suitable cutting device such as a scissors, scalpel, or knife.

The adhesive skin patch 1 can have any suitable length. In one embodiment of the present invention, the patch can be a self-wound roll 25 without a release liner 10 mounted on the front side 3 of the backing 2 of the adhesive skin patch 1. See, e.g., FIG. 10. In such an embodiment, the adhesive skin patch 1 can have a length of about 12 inches to about 100 yards, about 10 feet to about 50 yards, or about 20 feet to about 20 yards. Additionally, in such an embodiment, the adhesive skin patch 1 can have a width of about 0.1 inch to about 5.0 inches, about 0.1 inch to about 1.0 inch, or about 0.1 inch to about 0.5 inch.

In one embodiment of the present invention, the adhesive skin patch 1 can be rectangular and can have a release liner 10 mounted on the front side 3 of the backing 2 of the adhesive skin patch 1. In such an embodiment, the adhesive skin patch 1 can typically have a length of up to about 10 inches, up to about 6 inches, up to about 4 inches, or up to about 2 inches. The adhesive skin patch 1 can have any suitable width. Typically, the adhesive skin patch 1 will have a width of up to about 5 inches, up to about 2.5 inches, up to about 1 inch, or up to about 0.5 inch. Additionally, the adhesive skin patch 1 can have any suitable thickness. Typically, the adhesive skin patch 1 will have a thickness of about 0.10 mm to about 2.0 mm, about 0.15 mm to about 1.0 mm, or about 0.20 mm to about 0.75 mm.

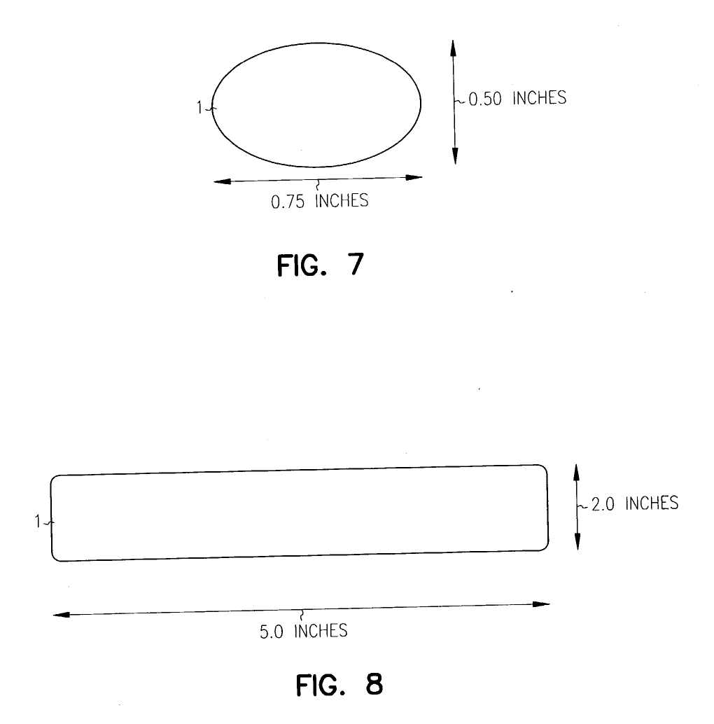

In one specific embodiment of the present invention, the adhesive skin patch 1 can be crescent, oval or circular in shape. The circular adhesive skin patch 1 can have a diameter of about 0.1 inch to about 10 inches. Preferably, the circular adhesive skin patch 1 can have a diameter of about 1.5 inches to about 5 inches. See, FIG. 7.

In another specific embodiment of the present invention, the adhesive skin patch 1 can be rectangular in shape. The rectangular adhesive skin patch 1 can have a glength of about 1 inch to about 10 inches and a width of about 1 inch to about 10 inches. Preferably, the rectangular adhesive skin patch 1 can have a length of about 1 inch to about 2 inches and a width of about 0.1 inch to about 0.75 inch. See, FIG. 8.

In one embodiment of the present invention, the adhesive skin patch 1 can have a release liner 10 mounted on the front side 3 of the backing 2 of the adhesive skin patch 1. In such an embodiment, one or more adhesive skin patches 1 can be mounted on the release liner 10. For example, one adhesive skin patch 1 can have one release liner 10 mounted on the front side 3 of the backing 2 of the adhesive skin patch 1. Alternatively, about 2 to about 100 or about 2 to about 20 adhesive skin patches 1 can be mounted on the release liner 10. The cost of having two or more patches 1 on a single release liner 10 is typically less expensive than skin patches 1 that are separately mounted on a single release liner 10. In addition, some consumers may prefer the ease and comfort of carrying a single patch assembly that includes a single release liner 10 and more than one (e.g., about 2 to about 20, or about 2 to about 10) adhesive patches 1 mounted on the single release liner 10.

The adhesive skin patch 1 can be applied to the region between the upper lip and the nose of a patient. Such a position will allow for the efficient flow of essential oil from the adhesive skin patch 1 to the nasal passageway of the patient. Alternatively, the adhesive skin patch 1 can be applied to an article of clothing of the patient. Specifically, the adhesive skin patch 1 can be applied to a mask (e.g., surgical mask), and the patient can wear the mask. While the adhesive skin patch 1 can be applied to either the inside or the outside of the mask, some patients may find it prefereable to apply to adhesive skin patch 1 to the inside of the mask. Such a location could efficiently create a zone of vapor concentration of the essential oil, in a known, discrete, effective, and safe amount. The zone being defined by that portion of the patient's face covered by the mask (which includes the patient's nasal passageway) and the mask itself. Such a zone of vapor concentration of the essential oil, in a known, discrete, effective, and safe amounts can be achieved in part, e.g., by utilizing the minimal inhibitory dosages (MIDs) disclosed in J. Antimicrobial Chemotherapy (2001) 47, 565-573.

The adhesive patch 1 of the present invention can be formulated or manufactured employing the above components. The adhesive patch 1 of the present invention can be formulated or manufactured using any suitable technique. Preferably, the adhesive patch 1 can be formulated or manufactured as described herein or as described in U.S. Pat. No. 5,536,263; U.S. Pat. No. 5,741,510; and references cited therein; wherein the backing can be treated with a sizing agent 8 prior to the infusion of the formulation 5.

Specific embodiments of the present invention are provided below:

[1] One embodiment of the present invention provides a method for preventing a respiratory infection in a mammal at risk thereof. The method includes contacting a live respiratory pathogen at risk of entering the respiratory tract of the mammal with a therapeutically effective amount of an essential oil, such that the live respiratory pathogen is inactivated upon contact with the essential oil, wherein the source of the essential oil is a patch located in the vicinity of the nasal passageway of the mammal.

[2] Another embodiment of the present invention provides a method for preventing a respiratory viral infection in a mammal at risk thereof. The method includes contacting a live respiratory virus with a prophylactically effective amount of an essential oil, such that the live respiratory virus is inactivated upon contact with the essential oil, wherein the source of the essential oil is a patch located in the vicinity of the nasal passageway of the mammal.

[3] Another embodiment of the present invention provides a method for preventing the transmission of a respiratory infection between mammals. The method includes contacting a live respiratory pathogen exiting the respiratory tract of a first mammal with a therapeutically effective amount of an essential oil, such that the live respiratory pathogen is inactivated upon contact with the essential oil, wherein the source of the essential oil is a patch located in the vicinity of the nasal passageway of the first mammal.

[4] Another embodiment of the present invention provides a method for inhibiting a respiratory pathogen. The method includes contacting a live respiratory pathogen with a therapeutically effective amount of an essential oil, such that the live respiratory pathogen is inactivated upon contact with the essential oil, wherein the source of the essential oil is a patch located in the vicinity of the respiratory pathogen.

[5] Another embodiment of the present invention provides a method for treating a respiratory infection in a mammal infected thereof or at risk thereof. The method includes contacting a live respiratory pathogen with a therapeutically effective amount of an essential oil, such that the live respiratory pathogen is inactivated upon contact with the essential oil, wherein the source of the essential oil is a patch located in the vicinity of the nasal passageway of the mammal.

[6] Another embodiment of the present invention provides a kit that includes: (a) patch that includes a flexible backing having a front side and a back side and a formulation positioned on at least a portion of the front side of the backing, in at least a portion of the front side of the backing, or on and in at least a portion of the front side of the backing, wherein the formulation includes a therapeutically effective respiratory pathogen inhibiting amount of an essential oil; (b) a mask for placing over the nasal passageway of a mammal; and (c) packaging material.

[7] Another embodiment of the present invention provides the kit of embodiment [6], further including instructions for using the patch and mask.

[8] Another embodiment of the present invention provides the method of any one of embodiments [1]-[5], wherein the patch includes a flexible backing having a front side and a back side and a formulation positioned on at least a portion of the front side of the backing, in at least a portion of the front side of the backing, or on and in at least a portion of the front side of the backing; wherein the formulation includes the essential oil.

[9] Another embodiment of the present invention provides the method of any one of embodiments [1]-[5] and [8], wherein the respiratory infection is an upper respiratory infection.

[10] Another embodiment of the present invention provides the method of any one of embodiments [1]-[5] and [8]-[9], wherein the respiratory infection is an acute respiratory infection.

[11] Another embodiment of the present invention provides the method of any one of embodiments [1]-[5] and [8]-[10], wherein the respiratory infection is a chronic respiratory infection.

[12] Another embodiment of the present invention provides the method of any one of embodiments [1]-[5] and [8]-[11], wherein the respiratory infection is caused by a respiratory virus.

[13] Another embodiment of the present invention provides the method of any one of embodiments [1]-[5] and [8]-[12], wherein the respiratory infection is selected from the group of severe acute respiratory syndrome (SARS), influenza, mumps, croup, sinusitis, bronchitis, angina, laryngitis, tracheitis, rhinitis, rhinopharyngitis, bronchiolitis, bronchitis, bronchopneumonia, pneumonia, staphylococcal pneumonia, whooping cough, the common cold, type A influenza, type B influenza, type C influenza, tuberculosis (TB), legionellosis, echinococcosis, pulmonary pleuropneumonia, tonsillitis, asthma, allergies, and combinations thereof.

[14] Another embodiment of the present invention provides the method of any one of embodiments [1]-[5] and [8]-[13], wherein the respiratory infection is caused by a pathogen selected from the group of respiratory syncytial virus (RSV), rhinovirus, para-influenza virus, coronavirus, adenovirus, coxsackievirus, myxovirus, Pneumococcus, Staphylococcus, Streptococcus, Klebsiella, Haemophi-lus, aspergillus, blastomyces dermatidis, candidiasis, coccidioidomycosis, cryptococcosis, histoplasmosis, contact allergens, and combinations thereof.

[15] Another embodiment of the present invention provides the method of any one of embodiments [1]-[14], wherein the backing of the patch is porous.

[16] Another embodiment of the present invention provides the method of any one of embodiments [1]-[14], wherein the backing of the patch is non-porous.

[17] Another embodiment of the present invention provides the method of any one of embodiments [1]-[14], wherein the backing of the patch is vapor permeable.

[18] Another embodiment of the present invention provides the method of any one of embodiments [1]-[17], wherein the formulation is positioned on the entire front side of the backing of the patch.

[19] Another embodiment of the present invention provides the method of any one of embodiments [1]-[17], wherein the formulation is positioned on a portion of front side of the backing of the patch.

[20] Another embodiment of the present invention provides the method of any one of embodiments [1]-[19], wherein the formulation is partially embedded in the front side of the backing of the patch.

[21] Another embodiment of the present invention provides the method of any one of embodiments [1]-[20], wherein the backing of the patch includes a non-woven fabric.

[22] Another embodiment of the present invention provides the method of any one of embodiments [1]-[20], wherein the backing of the patch includes polycellulose fibers, polyester fibers, polyurethane fibers, polyolefin fibers, polyamide fibers, cotton fibers, copolyester fibers, films, or any mixture thereof.

[23] Another embodiment of the present invention provides the method of any one of embodiments [1]-[20], wherein the backing of the patch includes open cell foam.

[24] Another embodiment of the present invention provides the method of embodiment [23], wherein the open cell foam includes polyurethane, polyvinyl chloride, polyethylene, or any combination thereof.

[25] Another embodiment of the present invention provides the method of any one of embodiments [1]-[24], wherein upon contact with skin, the backing of the patch retains the formulation and the patch allows moisture from the skin to pass through the patch.

[26] Another embodiment of the present invention provides the method of any one of embodiments [1]-[25], wherein the formulation further includes an adhesive.

[27] Another embodiment of the present invention provides the method of embodiment [26], wherein the adhesive is an acrylic ester copolymer, a water-based adhesive, a hot melt adhesive, a pressure sensitive adhesive, a solvent based pressure sensitive adhesive, a polyacrylate, a polyisobutylene, a polybutene, a rubber, a silicone based pressure sensitive adhesive, a polystyrene-polybutadiene-polystyrene block polymer, a polystyrene-polyisoprene-polystyrene block polymer, a polystyrene-poly(ethylene-butylene)-polystyrene block polymer, or any combination thereof.

[28] Another embodiment of the present invention provides the method of embodiment [26], wherein the adhesive is an acrylic ester copolymer.

[29] Another embodiment of the present invention provides the method of embodiment [28], wherein the acrylic ester copolymer is present in about 0.5 wt. % to about 18.0 wt. % of the formulation.

[30] Another embodiment of the present invention provides the method of any one of embodiments [1]-[29], wherein the formulation further includes a solvent.

[31] Another embodiment of the present invention provides the method of embodiment [30], wherein the solvent includes water; triethylene glycol; ethylene glycol; glycerin; propylene glycol; triacetin; 1,3-propane diol; 2-methyl-1,3-propane diol; glycerol ricinoleate; PEG-6 caprylic/capric glycerides; caprylic/capric triglycerides; propyleneglycol dicaprylate/dicaprate; glycerol monostearate; glycerol monocaprylate; glycerol monolaurate; neopentyl alcohol; 1-hexadecanol; hydroxypropyl beta-cyclodextrin; vitamin E; vitamin E acetate; deoxycholic acid; taurodeoxycholic acid; 3-[(3-cholamidopropyl) dimethylammonio]-1-propane-sulfonate; BigCHAP; cholic acid; cholesterol NF; propylene carbonate; lecithin; a pharmaceutically acceptable salt thereof; or a combination thereof.

[32] Another embodiment of the present invention provides the method of embodiment [30], wherein the solvent includes a (C₁-C₁₂) acyclic hydrocarbon, a (C₃-C₁₂) cyclic hydrocarbon, a (C₆-C₁₂) aryl hydrocarbon, a (C₆-C₁₂) heteroaryl hydrocarbon, or a (C₃-C₁₂) heterocyclic hydrocarbon;

wherein any of the hydrocarbons can optionally include one or more carbon-carbon double bonds and any of the hydrocarbons can optionally include one or more carbon- carbon triple bonds;

wherein any of the hydrocarbons can optionally include one or more oxy (—O—), carbonyl (—C(═O)C—), carboxylato (—C(═O)O—), dioxy (—O—O—), dithio (—S—S—), imino (—NH—), methylene dioxy (—OCH₂O—), sulfinyl (—SO—), sulfonyl (—SO₂—), or thio (—S—);

wherein any of the hydrocarbons can optionally be substituted with one or more amino, hydroxyl, cyano, nitro, (C₁-C₁₂)alkoxy, halo, trifluoro, trifluoro (C₁-C₁₂)alkyl, NR¹R², or COOR¹; wherein R¹ and R¹ are each independently hydrogen, a (C₁-C₁₂) acyclic hydrocarbon or a (C₃-C₁₂) cyclic hydrocarbon.

[33] Another embodiment of the present invention provides the method of embodiment [30], wherein the solvent is present in about 3.0 wt% to about 25.0 wt. % of the formulation.

[34] Another embodiment of the present invention provides the method of any one of embodiments [1]-[33], wherein the formulation further includes a polymer.

[35] Another embodiment of the present invention provides the method of embodiment [34], wherein the polymer is karaya, a polyacrlyamide, xanthan gum, guar gum, a natural polymer, a synthetic polymer, a hydrophilic polymer, a hydrocolloidal polymer, starch, a starch derivative, vinyl acetate copolymer, polyvinyl pyrrolidone, polyethylene oxide, algin, derivatives of algin, a polyacrylate, polymaleic acid, polymaleic anhydride, a polyurethane, a polyurea, gum acacia, locust bean gum, modified guar gum, maltodextrin, carboxymethyl cellulose, carboxypropyl cellulose, polyvinyl alcohol, poly AMPS, or any combination thereof.

[36] Another embodiment of the present invention provides the method of embodiment [34], wherein the polymer is karaya.

[37] Another embodiment of the present invention provides the method of embodiment [36], wherein the karaya is present in about 13 wt. % to about 52 wt. % of the formulation.

[38] Another embodiment of the present invention provides the method of embodiment [26], wherein the adhesive is positioned on the entire front side of the backing of the patch.

[39] Another embodiment of the present invention provides the method of embodiment [26], wherein the adhesive is positioned on a portion of front side of the backing of the patch.

[40] Another embodiment of the present invention provides the method of embodiment [26], wherein the adhesive is partially embedded in at least a portion of the backing of the patch.

[41] Another embodiment of the present invention provides the method of any one of embodiments [1]-[40], wherein the formulation is positioned on the entire front side of the backing of the patch.

[42] Another embodiment of the present invention provides the method of any one of embodiments [1]-[40], wherein the formulation is positioned on a portion of front side of the backing of the patch.

[43] Another embodiment of the present invention provides the method of any one of embodiments [1]-[42], wherein the formulation is partially embedded in at least a portion of the backing of the patch.

[44] Another embodiment of the present invention provides the method of any one of embodiments [1]-[43], wherein the formulation further includes a fragrance.

[45] Another embodiment of the present invention provides the method of embodiment [44], wherein the fragrance is a floral scent, a fruit scent, a plant leaf scent, or any combination thereof.

[46] Another embodiment of the present invention provides the method of embodiment [44], wherein the fragrance includes grape fragrance, musk fragrance, light vanilla fragrance, Jergens lotion fragrance, Vaseline Intensive Care fragrance, Nivea Lotion fragrance, Ivory Soap fragrance, amaretto fragrance, blueberry fragrance, coffee fragrance, egg nog fragrance, peanut butter fragrance, rum cake fragrance, honey almond fragrance, ginger bread house fragrance, coffee cake & spice fragrance, raspberry rose fragrance, sassafras fragrance, strawberry fragrance, grapefruit pink fragrance, home sweet fragrance, jeweled citrus fragrance, lemon, mango fragrance, mulberry fragrance, orange flower fragrance, passion fruit fragrance, pikaki fragrance, freesia fragrance, china rain fragrance, coconut fragrance, apple fragrance, baked bread fragrance, cornucopia fragrance, lemon chiffon fragrance, peppermint twist fragrance, white cake fragrance, cherry pie fragrance, sugar plum fragrance, plum fragrance, romantic fragrance, sea fresh fragrance, tea fragrance, green floral fragrance, honeydew fragrance, kiwi fragrance, lilac fragrance, may bouquet fragrance, neutralizer fragrance, patchouli fragrance, peach fragrance, pine apple blossom fragrance, chocolate mint fragrance, frankincense fragrance, baked apple pie fragrance, cappuccino fragrance, cran-apple fragrance, maple syrup fragrance, buttered popcorn fragrance, sugar cookie fragrance, cotton candy fragrance, cranberry cobbler fragrance, plumeria fragrance, rum fragrance, spring fever fragrance, watermelon fragrance, guava fragrance, honeysuckle fragrance, hyacinth fragrance, macadamia nut fragrance, melon fragrance, oakmoss fragrance, papaya fragrance, pear pineapple fragrance, blueberry fragrance, citrus-ginseng fragrance, garden dreams fragrance, banana creme pie fragrance, chocolate mint fragrance, cranberry fragrance, macadamia nut fragrance, pumpkin pie fragrance, chocolate German cake fragrance, banana nut bread fragrance, sweet potato pie fragrance, raspberry fragrance, sandalwood fragrance, spring flowers fragrance, ylang fragrance, heather fragrance, jasmine fragrance, lavender fragrance, magnolia fragrance, mountain air fragrance, orange essence fragrance, paradise fragrance, peony fragrance, alpine breeze fragrance, chamomile fragrance, clover fragrance, gardenia fragrance, or any combination thereof.

[47] Another embodiment of the present invention provides the method of any one of embodiments [1]-[46], wherein the essential oil includes ajowan, sweet almond oil, allspice, aloe vera oil, ammi visnaga (khella), amyris, angelica root, angelica seed, anise, anise seed, star anise, apricot kernel oil, absolute arnica, avocado oil, unrefined avocado oil, Copaiba balsam, balsam Peru genuine, balsam Peru oil, balsam peru liquid resin, balsam tolu, sweet french basil, basil, basil ct. methyl chavicol, lemon ct. citral basil, sweet ct. linalool basil, bay laurel, bay leaf, bay rum, bay leaf West Indies, bees wax, unrefined bees wax, benzoin absolute, benzoin resinoid, bergamot, mint bergamot, Italian bergamot oil, free bergaptene bergamot, birch, sweet birch, borage oil, boronia, butter, buchu leaf, cajeput, calamus, calendula oil, infused calendula oil, camellia oil, cannabis, caraway, caraway seed, cardamom, absolute carnation, carrot seed, high carotol carrot seed, carrot seed oil, cassia, cassis bud (black currant), castor oil, catnip, oil of catnip, cedarleaf, western red cedarleaf, cedarwood, Atlas cedarwood, Himalayan cedarwood, Virginia cedarwood, celery seed, chamomile, blue chamomile, German chamomile, Moroccan chamomile, Moroccan wild chamomile, Roman chamomile, champaca, cilantro, true cinnamon bark, cinnamon bark, cinnamon leaf, cinnamon cassia, cistus, citronella, Java citronella, ciste oil, artificial civet, clary sage, high sclareol clary sage, clementine, Italian clementine peel oil, clove, clove bud, clove leaf, cocoa, cocoa butter, unrefined cocoa butter, coconut oil, refined coconut oil, cognac, combava petitgrain, coriander, green coriander, cornmint, costus oil, cumin, cypress, davana oil, dill, dill weed, elemi, erigeron (fleabane), eucalyptus citriodora, eucalyptus globulus, lemon eucalyptus, fennel, sweet fennel, fenugreek, fir, Canada fir needle, Siberia fir needle, white fir needle, frankincense, India frankincense, Oman frankincense, galbanum oil, garlic, genet, geranium, geranium leaf, geranium rose, Bourbon geranium, Egyptian geranium, ginger, Cochin extra ginger, ginsing, Siberian ginsing, Korean ginsing, grapefruit, pink grapefruit, white grapefruit, grapeseed oil, hazelnut oil, helichrysum, helichrysum immortelle, Mad. helichrysum, Balkan helichrysum, Corsica helichrysum, France helichrysum, hemp oil, absolute honeysuckle, hyssop, hyssop decumbens, absolute immortelle, fragrant aster inula, Jamaican gold, unrefined Jamaican gold, jasmine, absolute jasmine, grandiflorum jasmine, sambac jasmine, jojoba oil, helio-carrot in jojoba, melissa in jojoba, absolute jonquille, juniper berry, Siberia juniper berry, Croatia juniper berry, lanolin, unrefined anhydrous lanolin, lantana camara, laurel nobilis, lavandin, abrialis lavandin, grosso lavandin, lavender, Oregon lavender, Bulgarian lavender, Russian lavender, high-altitude lavendar, wild-crafted lavender, lavendin, organic lavindin, lemon, lemongrass, lime, distilled lime, expressed lime, litsea, litsea cubeba, blue, pink and white lotus, macadamia oil, mace, green mandarin, red mandarin, yellow mandarin, manuka, absolute marigold, marigold flower, marjoram, Spanish marjoram, sweet marjoram (true), massoia bark, melissa, codistilled melissa, “rectified” melissa, true melissa, absolute mimosa, mimosa, monarda, mugwort, musk seed, myrrh, myrtle, absolute narcissus, neroli (orange blossom), niaouli, nutmeg, extra nutmeg, oakmoss, absolute oak moss, olibanum, absolute opopanax, bitter orange, blood orange, sweet orange, wild West Indian orange, oregano, orris root, concrete orris, osmanthus, palm oil, refined palm oil, palmarosa, paprika, parsley seed, patchouli, India patchouli oil, Indonesia patchouli oil, peanut, peanut oil, pecan oil, pennyroyal, pepper, black pepper, super black pepper, peppermint, India peppermint, USA baby mint peppermint, pet perfume, petitgrain (orange leaves), white pine, pine needle, evening primrose, ravensara anisata, true ravensara, ravensare, ravintsara, redberry, rosalina, rose, rose geranium, rose otto, Bulgarian rose, English rose, Turkish rose, rosehip seed oil, rosemary, rosemary anti-oxidant extract powder, rosemary verbenone, Morocco rosemary, Spain rosemary, rosewood, rosewood oil, rue, sage, white sage, sage dalmatian, sage officinalis, sage triloba, sandalwood, seabuckthorn berry, sesame oil, sesame seed oil, shea butter, unrefined shea butter, spikenard, green spikenard, spruce, St. John's wort, styrax resin, tagetes, tangerine, Dancy tangerine, tarragon, tea tree, Australia tea tree, thuja (cedar leaf), thyme, red thyme, thyme ct. linalool, thyme vulgaris, wild thyme, red thyme, mixed tocopherols, tolu balsam resin, absolute tuberose, tuberose, tumeric, valerian, vanilla, pure vanilla extract, vanilla bean, absolute vanilla bourbon, vegetable glycerin, absolute verbena, vetiver, violete leaves, vitex, organic Haiti vetiver, absolute violet leaf, walnut oil, wintergreen, natural wintergreen, wormwood, yarrow, ylang ylang, ylang ylang I, ylang ylang II, ylang ylang III, ylang ylang compound, ylang ylang complete, ylang ylang extra, methyl salicylate, menthol, camphor, eucalyptus oil, spearmint oil, or a combination thereof.

[48] Another embodiment of the present invention provides the method of any one of embodiments [1]-[46], wherein the essential oil includes basil (ocimum basilicum), cardamom (elettaria cardamomum), Roman chamomile (anthemis nobilis), eucalyptus (eucalyptus radiate), geranium (pelargonium x asperum), MQV (melaleuca quinquenervia viridiflora), neroli (citrus aurantium), petitgrain (citrus aurantium), rosemary (rosemarinus officinalis camphor), rosemary (rosemarinus officinalis verbenone), or a combination thereof.

[49] Another embodiment of the present invention provides the method of any one of embodiments [1]-[46], wherein the essential oil includes Satureja montana, carvacrol, thymol eugenol, australol, gaiacol, cinnamic aldehyde, geraniol, linalool, thujanol, myrcenol, terpineol, menthol and piperitol, geranial (citrals), citronellal, cuminal, verbenone, thujone, borneone (camphor), pinocamphone, cryptone, fenchone, menthone, piperitone and carvone, estragole, anethole, Phtalids (such as celery seed), cineole, monoterpenol, an essential oil derived from trees of the Myrteceae family; linalool oxide, linalool (Hissopus off. var. decumbens), thymol: Trachyspermum ammi (Ajowan), thymus CT thymol; caravcrol: Origanum compactum (Oregano) Origanum heracleoticum (Greek Oregano) Corydothymus capitatus (Spanish Oregano) Satureja montana (winter or mountain savory), thymus CT carvacrol, Thymus serpyllum (wild thyme or mother-of-thyme); Eugenol: Eugenia caryophyllus (Clove tree) Cinnamomum verum—leaf (Ceylon Cinnamon), ocimum gratissimum CT eugenol (hot or shrubby basil); gaiacol: guajacum officinalis (Gaiac wood); linalool: Aniba rosaeodora (rosewood), coriandrum sativum (Coriander), thymus CT linalool, Lavandula reydovan; Geraniol: Cymbopogon martini (Palmarosa), thymus CT geraniol; thujanol: Thymus CT thujanol, origanum majorana (Sweet marjoram or oregano); Borneol: Thyumus satureioides (Thym borneol-carvacrol type), Inula graveolens (sweet inula); menthol: Mentha x piperita (peppermint), Mentha arvensis (field mint or cornmint); Citronnellol: Pelargonium asperum (geranium); terpinenelol4: Melaleuca alternifolia (tea tree), origanum majorana (sweet marjoram or oregano); alpha terpineol: Ravensara aromatica (Ravensara), Eucalyptus radiata (black or narrow-leaf peppermint eucalyptus); Cinnamaldehyde: Cinnamomum verum or zeylandicum (bark) (Ceylon cinnamon (bark)), Cinnamomum cassia (bark) (Chinese cinnamon (bark), Cinnamomum loureirii (bark) (Vietnamese cinnamon bark); Cuminal: Cuminum cyminum (Cumin), Eucalyptus polybractea CT cryptone (Blue mallee eucalyptus cryptone type); Phellandral: eucalyptus polybractea CT cryptone (Blue Mallee eucalyptus cryptone type); or a combination thereof

[50] Another embodiment of the present invention provides the method of any one of embodiments [1]-[49], wherein at least a portion of the backing of the patch is treated with a sizing agent such that the portion of the backing that is treated with the sizing agent has a surface energy of about 20 dynes/cm² to about 65 dynes/cm².

[51] Another embodiment of the present invention provides the method of embodiment [50], wherein the sizing agent is a fluorocarbon solution, a silicone-containing compound, or a combination thereof.

[52] Another embodiment of the present invention provides the method of embodiment [51], wherein the fluorocarbon solution is Vilmed M1585 W/HY™, Vilmed M1585H/HY™, Vilmed M1586 W/HY™, Vilmed M1586 H/HY™, Vilmed M1570™, Vilmed M1573 F™, Vilmed M1573 FH™, Vilmed M1577 F™, Vilmed M1578 F™, Vilmed M1578 FH™, or a combination thereof.

[53] Another embodiment of the present invention provides the method of embodiment [51], wherein the silicone-containing compound is a polydimethyl siloxane, a dialkylsiloxane, a dimethylsiloxo vinyl alkene, a dialkylsiloxo vinyl alkene, a dimethylsiloxo acrylate, a dialkylsiloxo acrylate, a vinyl terminated polydimethylsiloxane, a vinyl terminated polydialkylsiloxane, or a combination thereof.

[54] Another embodiment of the present invention provides the method of embodiment [50], wherein the entire front side of the backing of the patch is treated with the sizing agent.

[55] Another embodiment of the present invention provides the method of embodiment [50], wherein the sizing agent penetrates at least a portion of the underlying surface of the front side of the backing of the patch.

[56] Another embodiment of the present invention provides the method of embodiment [50], wherein the sizing agent penetrates the entire underlying surface of the front side of the backing of the patch.

[57] Another embodiment of the present invention provides the method of embodiment [50], wherein the entire backing of the patch is treated with the sizing agent.

[58] Another embodiment of the present invention provides the method of any one of embodiments [1]-[57], wherein the patch further includes a skin protectant.

[59] Another embodiment of the present invention provides the method of embodiment [58], wherein the skin protectant is aloe, lanolin, glycerin, calamine, Vitamin E, Vitamin E acetate, Vitamin C, allantoin, aluminum hydroxide gel, bismuth subnitrate, boric acid, calamine, cocoa butter, dimethicone, glycerin, kaolin, live yeast cell derivative, petrolatum, pyridoxine hydrochloride, shark liver oil, sodium bicarbonate, sulfur, tannic acid, topical starch, trolamine, white petrolatum, zinc acetate, zinc carbonate zinc oxide, zinc sulfate, shea butter, or any combination thereof.

[60] Another embodiment of the present invention provides the method of any one of embodiments [1]-[59], wherein the formulation further includes a polyhydric alcohol.

[61] Another embodiment of the present invention provides the method of embodiment [60], wherein the polyhydric alcohol is erythritol, ethylene glycol, propylene glycol, triethylene glycol, tetraethylene glycol, sorbitol, or any combination thereof.

[62] Another embodiment of the present invention provides the method of embodiment [60], wherein the polyhydric alcohol is propylene glycol.

[63] Another embodiment of the present invention provides the method of embodiment [60], wherein the polyhydric alcohol is present in about 0.5 wt% to about 25.0 wt. % of the formulation.

[64] Another embodiment of the present invention provides the method of embodiment [60], wherein the polyhydric alcohol is present in about 0.5 wt% to about 5.0 wt. % of the formulation.

[65] Another embodiment of the present invention provides the method of any one of embodiments [1]-[64], wherein the formulation further includes water.

[66] Another embodiment of the present invention provides the method of embodiment [65], wherein the water is present in about 5.0 wt. % to about 15.0 wt. % of the formulation.

[67] Another embodiment of the present invention provides the method of any one of embodiments [1]-[66], wherein the formulation further includes an antibiotic agent.

[68] Another embodiment of the present invention provides the method of embodiment [67], wherein the antibiotic agent is cilastatin, clavulanic acid, folinic acid, probenecid, pyridoxine, sulbactam, dapsone, ethambutol, isoniazid, pyrazinamide, rifampin, streptomycin, capreomycin, ethionamide, para aminosalicylic acid, cycloserine, ciprofloxacin, nalidixic acid, norfloxacin, ofloxacin, imipenam, meropenem, cilistatin, cefadroxil, cefazolin, cephalexin, cephalothin, cefaclor, cefamandole, cefonicid, cefoxitin, cefuroxine, cefoperazone, cefotaxime, ceftazidime, ceftazidime, ceftizoxime, ceftriaxone, moxalactam, cefepine, bacitracin, vancomycin, aztreonam, amoxicillin, clavulanic acid, benzathine, penicillin g, penicillin v, ampicillin, carbenicillin indamyl, carbenicillin, mezlocillin, piperacillin, ticarcillin, cloxacillin, dicloxacillin, floxacillin, methicillin, nafcillin, oxacillin, colistmethate, polymixin b, trimethoprim, co-trimoxazole, mafenide, sulfadiazine, sodium sulfacetamide, sulfacytine, sulfadiazine, sulfamethoxazole , sulfapyridine, sulfasalazine, sulfisoxazole, chloramphenicol, clindamycin, spectinomycin, azithromycin, clarithromycin, erythrmoycin, erythromycin estolate, spiramycin, chlortetracycline, demeclocycline, doxycycline, minocycline, oxytetracycline, amikacin, kanamycin, neomycin, streptomycin, tobramycin, nitrofurantoin, griseofulvin, potassium iodide, fluconazole, itraconazole, ketoconazole, miconazole, clotrimazole, amphotericin b, nystatin, niclosamide, nifurtimox, piperazine, praziquantel, pyrantel pamoate, thiabendazole, amodiaquine, chloroquine, hydroxychloroquine, mefloquine, primaquine, pyrimethamine, quinidine gluconate, fansidar, diloxanide furoate, melarsoprol, nifurtimox, paromomycin, pentamidine, sodium stibogluconate, suramin, metronidazole, foscarnet, 3-deoxythmidin-2-ene, dideoxycytosine, dideoxyinosine, lamivudine, azidothymidine, indinavir, ritonavir, saquinavir, acyclovir, idoxuridine, ribavirin, vidarabine, amantidine, rinantidine, foscarnet, 3-deoxythmidin-2-ene, dideoxycytosine, dideoxyinosine, lamivudine, azidothymidine, indinavir, ritonavir, saquinavir, acyclovir, idoxuridine, ribavirin, vidarabine, amantidine, rinantidine, a pharmaceutically acceptable salt thereof, or any combination thereof.

[69] Another embodiment of the present invention provides the method of any one of embodiments [1]-[68], wherein the formulation further includes an antiviral agent selected from the group of Echinacea (Echinacea angustifolia, E. pallida, E. purpurea), Elderberry (Sambucus nigra), Garlic (Allium sativum), Lemon balm (Glycyrrhiza glabra), Astragalus (Astragalus membranaceus), eyebright (Euphrasia officinalis), sage (salvia officinalis), yarrow (Achillea millefolium), nettles (Urtica dioica), peppermint (menthe piperiya), ephedra (Ephedra sinica), marshmallow root (Althea officinalis), mullein leaves or flowers (Verbascum spp.), plantain leaf (Plantago lanceolata, P. major), licorice root, thyme (Thymus vulgaris), boneset (Eupatorium perfoliatum), feverfew (Tanacetum parthenium), catnip (Nepeta cataria), yarrow (Achillea millefolium), elder flower (Sambucus nigra, S. canadenis), ginger (Zingiber officinale), Ginko biloba, St. John's wort (Hypericum perforatum L.), and combinations thereof.

[70] Another embodiment of the present invention provides the method of any one of embodiments [1]-[68], wherein the formulation further includes an antiviral agent selected from the group of zinc, lysine, foscarnet, 3-deoxythmidin-2-ene, dideoxycytosine, dideoxyinosine, lamivudine, azidothymidine, indinavir, ritonavir, saquinavir, acyclovir, idoxuridine, ribavirin, vidarabine, amantidine, rinantidine, viracea2, cytovene, famciclovir, valaciclovir, penciclovir, hexadecylosypropyl-cidofovir (HDP-CDV), nonoxynol-9, docosanol (n-docosanol, 1-docosanol, or behenyl alcohol; which is a saturated 22-carbon straight-chain alcohol), a pharmaceutically acceptable salt thereof, and combinations thereof.

[71] Another embodiment of the present invention provides the method of any one of embodiments [1]-[68], wherein the formulation further includes an antiviral agent selected from the group of a hypochloride, a hypochloride generating compound, a peroxide, a peroxide generating compound, an organic halide, an organic halide generating compound, or a combination thereof.

[72] Another embodiment of the present invention provides the method of any one of embodiments [1]-[71], wherein the formulation further includes an antimicrobial agent.

[73] Another embodiment of the present invention provides the method of embodiment [72], wherein the antimicrobial agent is quat-15, a paraben, dichlorobenzyl alcohol, ethylene diamine tetreacetic acid, formaldehyde, gum benzoin, imidazolidinyl urea, phenyl-mercuric acetate, poly aminopropyl biguanide, proply gallate, sorbic acid, cresol, chloroacetamide sodium benzoate, chloromethyl-methylisothiazolinone, chloromethyl-methylisothiazolon, chloromethyl-methylisothiazolinone benzalkonium chloride, an octylisothiazolinone benzimidazol-compound, chloromethyl-methylisothiazolinone octylisothiazolinone, o-phenylphenol benzisothiazolinone, o-phenylphenol benzisothiazolinone, benzisothiazolinone, an aliphatic amine of 2-thiopyridineoxide, benzoic acid, editic acid, phenolic acid, benzyl alcohol, isopropyl alcohol, benzenethonium chloride, bronopol, cetrimide, chlorohexidine, chlorobutanol, chlorocresol, phenol, phenoxyethanol, phenyl ethyl alcohol, phenylmercuric acetate, phenylmercuric borate, phenylmercuric nitrate, potassium sorbate, proplyene glycol, sodium benzoate, sodium propionate, thimerosol, a pharmaceutically acceptable salt thereof, or any combination thereof.

[74] Another embodiment of the present invention provides the method of embodiment [72], wherein the antimicrobial agent is quat-15.

[75] Another embodiment of the present invention provides the method of embodiment [74], wherein the quat-15 is present in about 0.01 wt. % to about 0.1 wt. % of the formulation.

[76] Another embodiment of the present invention provides the method of any one of embodiments [1]-[75], wherein the patch is individually wrapped.

[77] Another embodiment of the present invention provides the method of any one of embodiments [1]-[76], wherein the patch further includes a release liner that is mounted to the front side of the backing.

[78] Another embodiment of the present invention provides the method of any one of embodiments [1]-[77], wherein the patch is sterile.

[79] Another embodiment of the present invention provides the method of embodiment [78], wherein the patch is sterilized by irradiation.

[80] Another embodiment of the present invention provides the method of embodiment [78], wherein the patch is sterilized by terminal irradiation.

[81] Another embodiment of the present invention provides the method of any one of embodiments [1]-[80], wherein the patch further includes packaging material.

[82] Another embodiment of the present invention provides the method of any one of embodiments [1]-[81], wherein the patch releases the therapeutically effective amount of the essential oil over a period of time of up to about 12 hours.

[83] Another embodiment of the present invention provides the method of any one of embodiments [1]-[81], wherein the patch releases the therapeutically effective amount of the essential oil over a period of time of up to about 8 hours.

[84] Another embodiment of the present invention provides the method of any one of embodiments [1]-[83], wherein the source of the essential oil is located within about 6 inches of the mammal.

[85] Another embodiment of the present invention provides the method of any one of embodiments [1]-[84], wherein the minimal inhibitory dose (MID) of the essential oil, as a measure of the vapor activity, is up to about 2.0 mg/L in air.

[86] Another embodiment of the present invention provides the method of any one of embodiments [1]-[84], wherein the minimal inhibitory dose (MID) of the essential oil, as a measure of the vapor activity, is about 0.1 mg/L to about 2.0 mg/L in air.

[87] Another embodiment of the present invention provides the method of any one of embodiments [1]-[86], wherein the source of the essential oil is located within about 6 inches of nasal passageway of the mammal.

The invention can be illustrated by the following non-limiting examples.

### EXAMPLES

### Example 1

| | Range % | Typical % |
|---|---|---|
| Glycerin | 20-70 | 50 |
| Karaya | 15-50 | 27 |
| aloe vera | 0.1-10 | 0.97 |
| acrylic emulsion adhesive | 2-30 | 14 |
| water | 1-20 | 2 |
| natural peppermint oil | 1-5 | 1 |
| eucalyptus oil | 1-5 | 1 |
| Spanish rosemary | 1-5 | 2 |
| sweet basil | 1-5 | 2 |
| Q-15 | 0.01-0.5 | 0.03 |

### Example 2

| | Range % | Typical % |
|---|---|---|
| Glycerin | 20-70 | 50 |
| pectin | 15-50 | 17 |
| polyacrylamide | 10-40 | 10 |
| aloe vera | 0.1-10 | 0.97 |
| acrylic emulsion adhesive | 2-30 | 14 |
| water | 1-20 | 2 |
| natural peppermint oil | 1-5 | 1 |
| eucalyptus oil | 1-5 | 1 |
| fennel | 1-5 | 2 |
| myrtle | 1-5 | 2 |
| Q-15 | 0.01-0.5 | 0.03 |

### Example 3

| | Range % | Typical % |
|---|---|---|
| Glycerin | 20-70 | 50 |
| pectin | 15-50 | 17 |
| polyacrylamide | 10-40 | 10 |
| aloe vera | 0.1-10 | 0.97 |
| acrylic emulsion adhesive | 2-30 | 14 |
| water | 1-20 | 2 |
| juniper berry oil | 1-5 | 1 |
| eucalyptus oil | 1-5 | 1 |
| fennel | 1-5 | 2 |
| myrtle | 1-5 | 2 |
| Q-15 | 0.01-0.5 | 0.03 |

### Example 4

| | Range % | Typical % |
|---|---|---|
| Glycerin | 20-70 | 50 |
| Karaya | 15-50 | 17 |
| Polyvinyl alcohol | 5-40 | 10 |
| aloe vera | 0.1-10 | 0.97 |
| acrylic emulsion adhesive | 2-30 | 14 |
| water | 1-20 | 2 |
| lavender | 1-6 | 3 |
| ylang ylang | 1-6 | 3 |
| Q-15 | 0.01-0.5 | 0.03 |

### Example 5

| | Range % | Typical % |
|---|---|---|
| Glycerin | 20-70 | 50 |
| Polyvinyl alcohol | 10-50 | 17 |
| Gelatin | 1-20 | 4 |
| aloe vera | 0.1-10 | 0.97 |
| acrylic emulsion adhesive | 2-30 | 14 |
| water | 1-20 | 8 |
| natural peppermint oil | 1-5 | 1 |
| eucalyptus oil | 1-5 | 1 |
| oregano | 1-5 | 2 |
| pine needle | 1-5 | 2 |
| Q-15 | 0.01-0.5 | 0.03 |

### Example 6

| | Range % | Typical % |
|---|---|---|
| Glycerin | 20-70 | 50 |
| Polyvinyl alcohol | 10-50 | 17 |
| Gelatin | 1-20 | 4 |
| aloe vera | 0.1-10 | 0.97 |
| acrylic emulsion adhesive | 2-30 | 14 |
| water | 1-20 | 8 |
| bergamot | 1-5 | 1 |
| lavender | 1-5 | 1 |
| clary sage | 1-5 | 2 |
| sweet marjoram | 1-5 | 2 |
| Q-15 | 0.01-0.5 | 0.03 |

### Example 7

| | Range % | Typical % |
|---|---|---|
| Glycerin | 20-70 | 50 |
| Polyvinyl alcohol | 10-40 | 10 |
| Gamma Cyclodextrin | 1-10 | 5 |
| Polyacrylamide | 5-30 | 12 |
| aloe vera | 0.1-10 | 0.97 |
| acrylic emulsion adhesive | 2-30 | 14 |
| water | 1-20 | 2 |
| natural peppermint oil | 1-5 | 1 |
| eucalyptus oil | 1-5 | 1 |
| Spanish rosemary | 1-5 | 2 |
| thyme | 1-5 | 2 |
| Q-15 | 0.01-0.5 | 0.03 |

### Example 8

| | Range % | Typical % |
|---|---|---|
| Glycerin | 20-70 | 50 |
| Polyvinyl alcohol | 10-40 | 10 |
| Gamma Cyclodextrin | 1-10 | 5 |
| Polyacrylamide | 5-30 | 12 |
| aloe vera | 0.1-10 | 0.97 |
| acrylic emulsion adhesive | 2-30 | 14 |
| water | 1-20 | 2 |
| natural peppermint oil | 1-5 | 1 |
| bergamot | 1-5 | 1 |
| Spanish rosemary | 1-5 | 2 |
| basil | 1-5 | 2 |
| Q-15 | 0.01-0.5 | 0.03 |

### Example 9

| | Range % | Typical % |
|---|---|---|
| Glycerin | 20-70 | 50 |
| Polyvinyl pyrrolidone | 15-50 | 17 |
| Polyvinyl alcohol | 10-30 | 7 |
| Beta-cyclodextrin | 1-10 | 3 |
| aloe vera | 0.1-10 | 0.97 |
| gelatin | 1-20 | 4 |
| water | 1-20 | 12 |
| natural peppermint oil | 1-5 | 1 |
| eucalyptus oil | 1-5 | 2 |
| cypress | 1-5 | 2 |
| cinnamon | 1-5 | 1 |
| Q-15 | 0.01-0.5 | 0.03 |

### Example 10

| | Range % | Typical % |
|---|---|---|
| Glycerin | 20-70 | 50 |
| algin | 15-50 | 17 |
| gum acacia | 5-30 | 5 |
| polyacrylic acid | 1-10 | 5 |
| aloe vera | 0.1-10 | 0.97 |
| acrylic emulsion adhesive | 2-30 | 4 |
| water with 2% multivalent salt | 1-20 | 12 |
| angelica root | 1-5 | 1 |
| bergamot | 1-5 | 1 |
| chamomile | 1-5 | 2 |
| geranium | 1-5 | 2 |
| Q-15 | 0.01-0.5 | 0.03 |

### Example 11

| | Range % | Typical % |
|---|---|---|
| Glycerin | 20-70 | 50 |
| Propylene glycol | 10-30 | 4 |
| polyacrylamide | 15-50 | 16 |
| polyvinyl alcohol | 15-40 | 13 |
| aloe vera | 0.1-10 | 0.97 |
| acrylic emulsion | 2-30 | 10 |
| adhesive | | |
| water | 1-20 | 2 |
| hyssop | 1-5 | 2 |
| lavender | 1-5 | 2 |
| Q-15 | 0.01-0.5 | 0.03 |

### Example 12

| | Range % | Typical % |
|---|---|---|
| Glycerin | 20-70 | 50 |
| polyacrylamide | 15-50 | 17 |
| malto dextrin | 5-25 | 10 |
| aloe vera | 0.1-10 | 0.97 |
| gelatin | 1-20 | 4 |
| water | 1-20 | 12 |
| natural peppermint | 1-5 | 1 |
| oil | | |
| eucalyptus oil | 1-5 | 1 |
| myrrh | 1-5 | 2 |
| pine needle | 1-5 | 2 |
| Q-15 | 0.01-0.5 | 0.03 |

### Example 13

| | Range % | Typical % |
|---|---|---|
| Glycerin | 20-70 | 50 |
| polyacrylamide | 15-50 | 17 |
| malto dextrin | 5-25 | 10 |
| aloe vera | 0.1-10 | 0.97 |
| gelatin | 1-20 | 4 |
| water | 1-20 | 12 |
| patchouli oil | 1-5 | 1 |
| eucalyptus oil | 1-5 | 1 |
| myrrh | 1-5 | 2 |
| pine needle | 1-5 | 2 |
| Q-15 | 0.01-0.5 | 0.03 |

### Example 14

| | Range % | Typical % |
|---|---|---|
| Glycerin | 20-70 | 40 |
| Propylene glycol | 10-30 | 5 |
| Karaya | 15-50 | 27 |
| aloe vera | 0.1-10 | 0.97 |
| gelatin | 1-20 | 5 |
| polyacrylic acid | 1-10 | 3 |
| water | 1-20 | 13 |
| rosewood | 1-5 | 1 |
| lavender | 1-5 | 1 |
| helichrysum | 1-5 | 2 |
| cedarwood | 1-5 | 2 |
| Q-15 | 0.01-0.5 | 0.03 |

### Example 15

| | Range % | Typical % |
|---|---|---|
| Glycerin | 20-70 | 50 |
| Karaya | 15-50 | 17 |
| Polyvinyl alcohol | 5-40 | 10 |
| aloe vera | 0.1-10 | 0.97 |
| acrylic emulsion | 2-30 | 14 |
| adhesive | | |
| water | 1-20 | 2 |
| camphor | 1-6 | 3 |
| menthol | 1-6 | 3 |
| Q-15 | 0.01-0.5 | 0.03 |

### Example 16

| | Range % | Typical % |
|---|---|---|
| Glycerin | 20-70 | 50 |
| polyacrylamide | 15-50 | 17 |
| malto dextrin | 5-25 | 10 |
| aloe vera | 0.1-10 | 0.97 |
| gelatin | 1-20 | 4 |
| water | 1-20 | 12 |
| camphor | 1-5 | 1 |
| menthol | 1-5 | 1 |
| myrrh | 1-5 | 2 |
| pine needle | 1-5 | 2 |
| Q-15 | 0.01-0.5 | 0.03 |

All publications, patents, and patent documents cited herein are incorporated by reference herein, as though individually incorporated by reference. The invention has been described with reference to various specific and preferred embodiments and techniques. However, it should be understood that many variations and modifications may be made while remaining within the spirit and scope of the invention.

1. A method for preventing a respiratory infection in a mammal at risk thereof, the method comprises contacting a live respiratory pathogen at risk of entering the respiratory tract of the mammal with a therapeutically effective amount of an essential oil, such that the live respiratory pathogen is inactivated upon contact with the essential oil, wherein the source of the essential oil is a patch located in the vicinity of the nasal passageway of the mammal.

2. The method of claim 1 wherein the patch comprises:
a flexible backing having a front side and a back side and a formulation positioned on at least a portion of the front side of the backing, in at least a portion of the front side of the backing, or on and in at least a portion of the front side of the backing;
wherein the formulation comprises the essential oil.

3. The method of claim 1 wherein the respiratory infection is an upper respiratory infection.

4. The method of claim 1 wherein the respiratory infection is an acute respiratory infection.

5. The method of claim 1 wherein the respiratory infection is a chronic respiratory infection.

6. The method of claim 1 wherein the respiratory infection is caused by a respiratory virus.

7. The method of claim 1 wherein the respiratory infection is selected from the group of severe acute respiratory syndrome (SARS), influenza, mumps, croup, sinusitis, bronchitis, angina, laryngitis, tracheitis, rhinitis, rhinopharyngitis, bronchiolitis, bronchitis, bronchopneumonia, pneumonia, staphylococcal pneumonia, whooping cough, the common cold, type A influenza, type B influenza, type C influenza, tuberculosis (TB), legionellosis, echinococcosis, pulmonary pleuropneumonia, tonsillitis, asthma, allergies, and combinations thereof.

8. The method of claim 1 wherein the respiratory infection is caused by a pathogen selected from the group of respiratory syncytial virus (RSV), rhinovirus, para-influenza virus, coronavirus, adenovirus, coxsackievirus, myxovirus, Pneumococcus, Staphylococcus, Streptococcus, Klebsiella, Haemophi-lus, aspergillus, blastomyces dermatidis, candidiasis, coccidioidomycosis, cryptococcosis, histoplasmosis, contact allergens, and combinations thereof.

9. The method of claim 2 wherein the backing of the patch is porous.

10. The method of claim 2 wherein the backing of the patch is non-porous.

11. The method of claim 2 wherein the backing of the patch is vapor permeable.

12. The method of claim 2 wherein the formulation is positioned on the entire front side of the backing of the patch.

13. The method of claim 2 wherein the formulation is positioned on a portion of front side of the backing of the patch.

14. The method of claim 2 wherein the formulation is partially embedded in the front side of the backing of the patch.

15. The method of claim 2 wherein the backing of the patch comprises a non-woven fabric.

16. The method of claim 2 wherein the backing of the patch comprises polycellulose fibers, polyester fibers, polyurethane fibers, polyolefin fibers, polyamide fibers, cotton fibers, copolyester fibers, films, or any mixture thereof.

17. The method of claim 2 wherein the backing of the patch comprises open cell foam.

18. The method of claim 17 wherein the open cell foam comprises polyurethane, polyvinyl chloride, polyethylene, or any combination thereof.

19. The method of claim 2 wherein upon contact with skin, the backing of the patch retains the formulation and the patch allows moisture from the skin to pass through the patch.

20. The method of claim 2 wherein the formulation further comprises an adhesive.

21. The method of claim 20 wherein the adhesive is an acrylic ester copolymer, a water-based adhesive, a hot melt adhesive, a pressure sensitive adhesive, a solvent based pressure sensitive adhesive, a polyacrylate, a polyisobutylene, a polybutene, a rubber, a silicone based pressure sensitive adhesive, a polystyrene-polybutadiene-polystyrene block polymer, a polystyrene-polyisoprene-polystyrene block polymer, a polystyrene-poly(ethylene-butylene)-polystyrene block polymer, or any combination thereof.

22. The method of claim 20 wherein the adhesive is an acrylic ester copolymer.

23. The method of claim 22 wherein the acrylic ester copolymer is present in about 0.5 wt. % to about 18.0 wt. % of the formulation.

24. The method of claim 2 wherein the formulation further comprises a solvent.

25. The method of claim 24 wherein the solvent comprises water; triethylene glycol; ethylene glycol; glycerin; propylene glycol; triacetin; 1,3-propane diol; 2-methyl-1,3-propane diol; glycerol ricinoleate; PEG-6 caprylic/capric glycerides; caprylic/capric triglycerides; propyleneglycol dicaprylate/dicaprate; glycerol monostearate; glycerol monocaprylate; glycerol monolaurate; neopentyl alcohol; 1-hexadecanol; hydroxypropyl beta-cyclodextrin; vitamin E; vitamin E acetate; deoxycholic acid; taurodeoxycholic acid; 3-[(3-cholamidopropyl) dimethylammonio]-1-propane-sulfonate; BigCHAP; cholic acid; cholesterol NF; propylene carbonate; lecithin; a pharmaceutically acceptable salt thereof; or a combination thereof.

26. The method of claim 24 wherein the solvent comprises a (C₁-C₁₂) acyclic hydrocarbon, a (C₃-C₁₂) cyclic hydrocarbon, a (C₆-C₁₂) aryl hydrocarbon, a (C₆-C₁₂) heteroaryl hydrocarbon, or a (C₃-C₁₂) heterocyclic hydrocarbon;
wherein any of the hydrocarbons can optionally include one or more carbon-carbon double bonds and any of the hydrocarbons can optionally include one or more carbon-carbon triple bonds;
wherein any of the hydrocarbons can optionally include one or more oxy (—O—), carbonyl (—C(═O)C—), carboxylato (—C(═O)O—), dioxy (—O—O—), dithio (—S—S—), imino (—NH—), methylene dioxy (—OCH₂O—), sulfinyl (—SO—), sulfonyl (—SO₂—), or thio (—S—);
wherein any of the hydrocarbons can optionally be substituted with one or more amino, hydroxyl, cyano, nitro, (C₁-C₁₂)alkoxy, halo, trifluoro, trifluoro (C₁-C₁₂)alkyl, NR¹R², or COOR¹; wherein R¹ and R² are each independently hydrogen, a (C₁-C₁₂) acyclic hydrocarbon or a (C₃-C₁₂) cyclic hydrocarbon.

27. The method of claim 24 wherein the solvent is present in about 3.0 wt% to about 25.0 wt. % of the formulation.

28. The method of claim 2 wherein the formulation further comprises a polymer.

29. The method of claim 28 wherein the polymer is karaya, a polyacrlyamide, xanthan gum, guar gum, a natural polymer, a synthetic polymer, a hydrophilic polymer, a hydrocolloidal polymer, starch, a starch derivative, vinyl acetate copolymer, polyvinyl pyrrolidone, polyethylene oxide, algin, derivatives of algin, a polyacrylate, polymaleic acid, polymaleic anhydride, a polyurethane, a polyurea, gum acacia, locust bean gum, modified guar gum, maltodextrin, carboxymethyl cellulose, carboxypropyl cellulose, polyvinyl alcohol, poly AMPS, or any combination thereof.

30. The method of claim 28 wherein the polymer is karaya.

31. The method of claim 30 wherein the karaya is present in about 13 wt. % to about 52 wt. % of the formulation.

32. The method of claim 20 wherein the adhesive is positioned on the entire front side of the backing of the patch.

33. The method of claim 20 wherein the adhesive is positioned on a portion of front side of the backing of the patch.

34. The method of claim 20 wherein the adhesive is partially embedded in at least a portion of the backing of the patch.

35. The method of claim 2 wherein the formulation is positioned on the entire front side of the backing of the patch.

36. The method of claim 2 wherein the formulation is positioned on a portion of front side of the backing of the patch.

37. The method of claim 2 wherein the formulation is partially embedded in at least a portion of the backing of the patch.

38. The method of claim 2 wherein the formulation further comprises a fragrance.

39. The method of claim 38 wherein the fragrance is a floral scent, a fruit scent, a plant leaf scent, or any combination thereof.

40. The method of claim 38 wherein the fragrance comprises grape fragrance, musk fragrance, light vanilla fragrance, Jergens lotion fragrance, Vaseline Intensive Care fragrance, Nivea Lotion fragrance, Ivory Soap fragrance, amaretto fragrance, blueberry fragrance, coffee fragrance, egg nog fragrance, peanut butter fragrance, rum cake fragrance, honey almond fragrance, ginger bread house fragrance, coffee cake & spice fragrance, raspberry rose fragrance, sassafras fragrance, strawberry fragrance, grapefruit pink fragrance, home sweet fragrance, jeweled citrus fragrance, lemon, mango fragrance, mulberry fragrance, orange flower fragrance, passion fruit fragrance, pikaki fragrance, freesia fragrance, china rain fragrance, coconut fragrance, apple fragrance, baked bread fragrance, cornucopia fragrance, lemon chiffon fragrance, peppermint twist fragrance, white cake fragrance, cherry pie fragrance, sugar plum fragrance, plum fragrance, romantic fragrance, sea fresh fragrance, tea fragrance, green floral fragrance, honeydew fragrance, kiwi fragrance, lilac fragrance, may bouquet fragrance, neutralizer fragrance, patchouli fragrance, peach fragrance, pine apple blossom fragrance, chocolate mint fragrance, frankincense fragrance, baked apple pie fragrance, cappuccino fragrance, cran-apple fragrance, maple syrup fragrance, buttered popcorn fragrance, sugar cookie fragrance, cotton candy fragrance, cranberry cobbler fragrance, plumeria fragrance, rum fragrance, spring fever fragrance, watermelon fragrance, guava fragrance, honeysuckle fragrance, hyacinth fragrance, macadamia nut fragrance, melon fragrance, oakmoss fragrance, papaya fragrance, pear pineapple fragrance, blueberry fragrance, citrus-ginseng fragrance, garden dreams fragrance, banana creme pie fragrance, chocolate mint fragrance, cranberry fragrance, macadamia nut fragrance, pumpkin pie fragrance, chocolate German cake fragrance, banana nut bread fragrance, sweet potato pie fragrance, raspberry fragrance, sandalwood fragrance, spring flowers fragrance, ylang fragrance, heather fragrance, jasmine fragrance, lavender fragrance, magnolia fragrance, mountain air fragrance, orange essence fragrance, paradise fragrance, peony fragrance, alpine breeze fragrance, chamomile fragrance, clover fragrance, gardenia fragrance, or any combination thereof.

41. The method of claim 2 wherein the essential oil comprises ajowan, sweet almond oil, allspice, aloe vera oil, ammi visnaga (khella), amyris, angelica root, angelica seed, anise, anise seed, star anise, apricot kernel oil, absolute arnica, avocado oil, unrefined avocado oil, Copaiba balsam, balsam Peru genuine, balsam Peru oil, balsam peru liquid resin, balsam tolu, sweet french basil, basil, basil ct. methyl chavicol, lemon ct. citral basil, sweet ct. linalool basil, bay laurel, bay leaf, bay rum, bay leaf West Indies, bees wax, unrefined bees wax, benzoin absolute, benzoin resinoid, bergamot, mint bergamot, Italian bergamot oil, free bergaptene bergamot, birch, sweet birch, borage oil, boronia, butter, buchu leaf, cajeput, calamus, calendula oil, infused calendula oil, camellia oil, cannabis, caraway, caraway seed, cardamom, absolute carnation, carrot seed, high carotol carrot seed, carrot seed oil, cassia, cassis bud (black currant), castor oil, catnip, oil of catnip, cedarleaf, western red cedarleaf, cedarwood, Atlas cedarwood, Himalayan cedarwood, Virginia cedarwood, celery seed, chamomile, blue chamomile, German chamomile, Moroccan chamomile, Moroccan wild chamomile, Roman chamomile, champaca, cilantro, true cinnamon bark, cinnamon bark, cinnamon leaf, cinnamon cassia, cistus, citronella, Java citronella, ciste oil, artificial civet, clary sage, high sclareol clary sage, clementine, Italian clementine peel oil, clove, clove bud, clove leaf, cocoa, cocoa butter, unrefined cocoa butter, coconut oil, refined coconut oil, cognac, combava petitgrain, coriander, green coriander, cornmint, costus oil, cumin, cypress, davana oil, dill, dill weed, elemi, erigeron (fleabane), eucalyptus citriodora, eucalyptus globulus, lemon eucalyptus, fennel, sweet fennel, fenugreek, fir, Canada fir needle, Siberia fir needle, white fir needle, frankincense, India frankincense, Oman frankincense, galbanum oil, garlic, genet, geranium, geranium leaf, geranium rose, Bourbon geranium, Egyptian geranium, ginger, Cochin extra ginger, ginsing, Siberian ginsing, Korean ginsing, grapefruit, pink grapefruit, white grapefruit, grapeseed oil, hazelnut oil, helichrysum, helichrysum immortelle, Mad. helichrysum, Balkan helichrysum, Corsica helichrysum, France helichrysum, hemp oil, absolute honeysuckle, hyssop, hyssop decumbens, absolute immortelle, fragrant aster inula, Jamaican gold, unrefined Jamaican gold, jasmine, absolute jasmine, grandiflorum jasmine, sambac jasmine, jojoba oil, helio-carrot in jojoba, melissa in jojoba, absolute jonquille, juniper berry, Siberia juniper berry, Croatia juniper berry, lanolin, unrefined anhydrous lanolin, lantana camara, laurel nobilis, lavandin, abrialis lavandin, grosso lavandin, lavender, Oregon lavender, Bulgarian lavender, Russian lavender, high-altitude lavendar, wild-crafted lavender, lavendin, organic lavindin, lemon, lemongrass, lime, distilled lime, expressed lime, litsea, litsea cubeba, blue, pink and white lotus, macadamia oil, mace, green mandarin, red mandarin, yellow mandarin, manuka, absolute marigold, marigold flower, marjoram, Spanish marjoram, sweet marjoram (true), massoia bark, melissa, codistilled melissa, “rectified” melissa, true melissa, absolute mimosa, mimosa, monarda, mugwort, musk seed, myrrh, myrtle, absolute narcissus, neroli (orange blossom), niaouli, nutmeg, extra nutmeg, oakmoss, absolute oak moss, olibanum, absolute opopanax, bitter orange, blood orange, sweet orange, wild West Indian orange, oregano, orris root, concrete orris, osmanthus, palm oil, refined palm oil, palmarosa, paprika, parsley seed, patchouli, India patchouli oil, Indonesia patchouli oil, peanut, peanut oil, pecan oil, pennyroyal, pepper, black pepper, super black pepper, peppermint, India peppermint, USA baby mint peppermint, pet perfume, petitgrain (orange leaves), white pine, pine needle, evening primrose, ravensara anisata, true ravensara, ravensare, ravintsara, redberry, rosalina, rose, rose geranium, rose otto, Bulgarian rose, English rose, Turkish rose, rosehip seed oil, rosemary, rosemary anti-oxidant extract powder, rosemary verbenone, Morocco rosemary, Spain rosemary, rosewood, rosewood oil, rue, sage, white sage, sage dalmatian, sage officinalis, sage triloba, sandalwood, seabuckthorn berry, sesame oil, sesame seed oil, shea butter, unrefined shea butter, spikenard, green spikenard, spruce, St. John's wort, styrax resin, tagetes, tangerine, Dancy tangerine, tarragon, tea tree, Australia tea tree, thuja (cedar leaf), thyme, red thyme, thyme ct. linalool, thyme vulgaris, wild thyme, red thyme, mixed tocopherols, tolu balsam resin, absolute tuberose, tuberose, tumeric, valerian, vanilla, pure vanilla extract, vanilla bean, absolute vanilla bourbon, vegetable glycerin, absolute verbena, vetiver, violete leaves, vitex, organic Haiti vetiver, absolute violet leaf, walnut oil, wintergreen, natural wintergreen, wormwood, yarrow, ylang ylang, ylang ylang I, ylang ylang II, ylang ylang III, ylang ylang compound, ylang ylang complete, ylang ylang extra, methyl salicylate, menthol, camphor, eucalyptus oil, spearmint oil, or a combination thereof.

42. The method of claim 2 wherein the essential oil comprises basil (ocimum basilicum), cardamom (elettaria cardamomum), Roman chamomile (anthemis nobilis), eucalyptus (eucalyptus radiate), geranium (pelargonium x asperum), MQV (melaleuca quinquenervia viridiflora), neroli (citrus aurantium), petitgrain (citrus aurantium), rosemary (rosemarinus officinalis camphor), rosemary (rosemarinus officinalis verbenone), or a combination thereof.

43. The method of claim 2 wherein the essential oil comprises Satureja montana, carvacrol, thymol eugenol, australol, gaiacol, cinnamic aldehyde, geraniol, linalool, thujanol, myrcenol, terpineol, menthol and piperitol, geranial (citrals), citronellal, cuminal, verbenone, thujone, borneone (camphor), pinocamphone, cryptone, fenchone, menthone, piperitone and carvone, estragole, anethole, Phtalids (such as celery seed), cineole, monoterpenol, an essential oil derived from trees of the Myrteceae family; linalool oxide, linalool (Hissopus off. var. decumbens), thymol: Trachyspermum ammi (Ajowan), thymus CT thymol; caravcrol: Origanum compactum (Oregano) Origanum heracleoticum (Greek Oregano) Corydothymus capitatus (Spanish Oregano) Satureja montana (winter or mountain savory), thymus CT carvacrol, Thymus serpyllum (wild thyme or mother-of-thyme); Eugenol: Eugenia caryophyllus (Clove tree) Cinnamomum verum—leaf (Ceylon Cinnamon), ocimum gratissimum CT eugenol (hot or shrubby basil); gaiacol: guajacum officinalis (Gaiac wood); linalool: Aniba rosaeodora (rosewood), coriandrum sativum (Coriander), thymus CT linalool, Lavandula reydovan; Geraniol: Cymbopogon martini (Palmarosa), thymus CT geraniol; thujanol: Thymus CT thujanol, origanum majorana (Sweet marjoram or oregano); Borneol: Thyumus satureioides (Thym borneol-carvacrol type), Inula graveolens (sweet inula); menthol: Mentha x piperita (peppermint), Mentha arvensis (field mint or cornmint); Citronnellol: Pelargonium asperum (geranium); terpinenelol4: Melaleuca alternifolia (tea tree), origanum majorana (sweet marjoram or oregano); alpha terpineol: Ravensara aromatica (Ravensara), Eucalyptus radiata (black or narrow-leaf peppermint eucalyptus); Cinnamaldehyde: Cinnamomum verum or zeylandicum (bark) (Ceylon cinnamon (bark)), Cinnamomum cassia (bark) (Chinese cinnamon (bark), Cinnamomum loureirii (bark) (Vietnamese cinnamon bark); Cuminal: Cuminum cyminum (Cumin), Eucalyptus polybractea CT cryptone (Blue mallee eucalyptus cryptone type); Phellandral: eucalyptus polybractea CT cryptone (Blue Mallee eucalyptus cryptone type); or a combination thereof

44. The method of claim 2 wherein at least a portion of the backing of the patch is treated with a sizing agent such that the portion of the backing that is treated with the sizing agent has a surface energy of about 20 dynes/cm² to about 65 dynes/cm².

45. The method of claim 44 wherein the sizing agent is a fluorocarbon solution, a silicone-containing compound, or a combination thereof.

46. The method of claim 45 wherein the fluorocarbon solution is Vilmed M1585 W/HY™, Vilmed M1585H/HY™, Vilmed M1586 W/HY™, Vilmed M1586 H/HY™, Vilmed M1570™, Vilmed M1573 F™, Vilmed M1573 FH™, Vilmed M1577 F™, Vilmed M1578 F™, Vilmed M1578 FH™, or a combination thereof.

47. The method of claim 45 wherein the silicone-containing compound is a polydimethyl siloxane, a dialkylsiloxane, a dimethylsiloxo vinyl alkene, a dialkylsiloxo vinyl alkene, a dimethylsiloxo acrylate, a dialkylsiloxo acrylate, a vinyl terminated polydimethylsiloxane, a vinyl terminated polydialkylsiloxane, or a combination thereof.

48. The method of claim 44 wherein the entire front side of the backing of the patch is treated with the sizing agent.

49. The method of claim 44 wherein the sizing agent penetrates at least a portion of the underlying surface of the front side of the backing of the patch.

50. The method of claim 44 wherein the sizing agent penetrates the entire underlying surface of the front side of the backing of the patch.

51. The method of claim 44 wherein the entire backing of the patch is treated with the sizing agent.

52. The method of claim 1 wherein the patch further comprises a skin protectant.

53. The method of claim 52 wherein the skin protectant is aloe, lanolin, glycerin, calamine, Vitamin E, Vitamin E acetate, Vitamin C, allantoin, aluminum hydroxide gel, bismuth subnitrate, boric acid, calamine, cocoa butter, dimethicone, glycerin, kaolin, live yeast cell derivative, petrolatum, pyridoxine hydrochloride, shark liver oil, sodium bicarbonate, sulfur, tannic acid, topical starch, trolamine, white petrolatum, zinc acetate, zinc carbonate zinc oxide, zinc sulfate, shea butter, or any combination thereof.

54. The method of claim 2 wherein the formulation further comprises a polyhydric alcohol.

55. The method of claim 54 wherein the polyhydric alcohol is erythritol, ethylene glycol, propylene glycol, triethylene glycol, tetraethylene glycol, sorbitol, or any combination thereof.

56. The method of claim 54 wherein the polyhydric alcohol is propylene glycol.

57. The method of claim 54 wherein the polyhydric alcohol is present in about 0.5 wt% to about 25.0 wt. % of the formulation.

58. The method of claim 54 wherein the polyhydric alcohol is present in about 0.5 wt% to about 5.0 wt. % of the formulation.

59. The method of claim 2 wherein the formulation further comprises water.

60. The method of claim 59 wherein the water is present in about 5.0 wt. % to about 15.0 wt. % of the formulation.

61. The method of claim 2 wherein the formulation further comprises an antibiotic agent.

62. The method of claim 61 wherein the antibiotic agent is cilastatin, clavulanic acid, folinic acid, probenecid, pyridoxine, sulbactam, dapsone, ethambutol, isoniazid, pyrazinamide, rifampin, streptomycin, capreomycin, ethionamide, para aminosalicylic acid, cycloserine, ciprofloxacin, nalidixic acid, norfloxacin, ofloxacin, imipenam, meropenem, cilistatin, cefadroxil, cefazolin, cephalexin, cephalothin, cefaclor, cefamandole, cefonicid, cefoxitin, cefuroxine, cefoperazone, cefotaxime, ceftazidime, ceftazidime, ceftizoxime, ceftriaxone, moxalactam, cefepine, bacitracin, vancomycin, aztreonam, amoxicillin, clavulanic acid, benzathine, penicillin g, penicillin v, ampicillin, carbenicillin indamyl, carbenicillin, mezlocillin, piperacillin, ticarcillin, cloxacillin, dicloxacillin, floxacillin, methicillin, nafcillin, oxacillin, colistmethate, polymixin b, trimethoprim, co-trimoxazole, mafenide, sulfadiazine, sodium sulfacetamide, sulfacytine, sulfadiazine, sulfamethoxazole , sulfapyridine, sulfasalazine, sulfisoxazole, chloramphenicol, clindamycin, spectinomycin, azithromycin, clarithromycin, erythrmoycin, erythromycin estolate, spiramycin, chlortetracycline, demeclocycline, doxycycline, minocycline, oxytetracycline, amikacin, kanamycin, neomycin, streptomycin, tobramycin, nitrofurantoin, griseofulvin, potassium iodide, fluconazole, itraconazole, ketoconazole, miconazole, clotrimazole, amphotericin b, nystatin, niclosamide, nifurtimox, piperazine, praziquantel, pyrantel pamoate, thiabendazole, amodiaquine, chloroquine, hydroxychloroquine, mefloquine, primaquine, pyrimethamine, quinidine gluconate, fansidar, diloxanide furoate, melarsoprol, nifurtimox, paromomycin, pentamidine, sodium stibogluconate, suramin, metronidazole, foscarnet, 3-deoxythmidin-2-ene, dideoxycytosine, dideoxyinosine, lamivudine, azidothymidine, indinavir, ritonavir, saquinavir, acyclovir, idoxuridine, ribavirin, vidarabine, amantidine, rinantidine, foscarnet, 3-deoxythmidin-2-ene, dideoxycytosine, dideoxyinosine, lamivudine, azidothymidine, indinavir, ritonavir, saquinavir, acyclovir, idoxuridine, ribavirin, vidarabine, amantidine, rinantidine, a pharmaceutically acceptable salt thereof, or any combination thereof.

63. The method of claim 2 wherein the formulation further comprises an antiviral agent selected from the group of Echinacea (Echinacea angustifolia, E. pallida, E. purpurea), Elderberry (Sambucus nigra), Garlic (Allium sativum), Lemon balm (Glycyrrhiza glabra), Astragalus (Astragalus membranaceus), eyebright (Euphrasia officinalis), sage (salvia officinalis), yarrow (Achillea millefolium), nettles (Urtica dioica), peppermint (menthe piperiya), ephedra (Ephedra sinica), marshmallow root (Althea officinalis), mullein leaves or flowers (Verbascum spp.), plantain leaf (Plantago lanceolata, P. major), licorice root, thyme (Thymus vulgaris), boneset (Eupatorium perfoliatum), feverfew (Tanacetum parthenium), catnip (Nepeta cataria), yarrow (Achillea millefolium), elder flower (Sambucus nigra, S. canadenis), ginger (Zingiber officinale), Ginko biloba, St. John's wort (Hypericum perforatum L.), and combinations thereof.

64. The method of claim 2 wherein the formulation further comprises an antiviral agent selected from the group of zinc, lysine, foscarnet, 3-deoxythmidin-2-ene, dideoxycytosine, dideoxyinosine, lamivudine, azidothymidine, indinavir, ritonavir, saquinavir, acyclovir, idoxuridine, ribavirin, vidarabine, amantidine, rinantidine, viracea2, cytovene, famciclovir, valaciclovir, penciclovir, hexadecylosypropyl-cidofovir (HDP-CDV), nonoxynol-9, docosanol (n-docosanol, 1-docosanol, or behenyl alcohol; which is a saturated 22-carbon straight-chain alcohol), a pharmaceutically acceptable salt thereof, and combinations thereof.

65. The method of claim 2 wherein the formulation further comprises an antiviral agent selected from the group of a hypochloride, a hypochloride generating compound, a peroxide, a peroxide generating compound, an organic halide, an organic halide generating compound, or a combination thereof.

66. The method of claim 2 wherein the formulation further comprises an antimicrobial agent.

67. The method of claim 66 wherein the antimicrobial agent is quat-15, a paraben, dichlorobenzyl alcohol, ethylene diamine tetreacetic acid, formaldehyde, gum benzoin, imidazolidinyl urea, phenyl-mercuric acetate, poly aminopropyl biguanide, proply gallate, sorbic acid, cresol, chloroacetamide sodium benzoate, chloromethyl-methylisothiazolinone, chloromethyl-methylisothiazolon, chloromethyl-methylisothiazolinone benzalkonium chloride, an octylisothiazolinone benzimidazol-compound, chloromethyl-methylisothiazolinone octylisothiazolinone, o-phenylphenol benzisothiazolinone, o-phenylphenol benzisothiazolinone, benzisothiazolinone, an aliphatic amine of 2-thiopyridineoxide, benzoic acid, editic acid, phenolic acid, benzyl alcohol, isopropyl alcohol, benzenethonium chloride, bronopol, cetrimide, chlorohexidine, chlorobutanol, chlorocresol, phenol, phenoxyethanol, phenyl ethyl alcohol, phenylmercuric acetate, phenylmercuric borate, phenylmercuric nitrate, potassium sorbate, proplyene glycol, sodium benzoate, sodium propionate, thimerosol, a pharmaceutically acceptable salt thereof, or any combination thereof.

68. The method of claim 66 wherein the antimicrobial agent is quat-15.

69. The method of claim 68 wherein the quat-15 is present in about 0.01 wt. % to about 0.1 wt. % of the formulation.

70. The method of claim 2 wherein the patch is individually wrapped.

71. The method of claim 2 wherein the patch further comprises a release liner that is mounted to the front side of the backing.

72. The method of claim 2 wherein the patch is sterile.

73. The method of claim 72 wherein the patch is sterilized by irradiation.

74. The method of claim 72 wherein the patch is sterilized by terminal irradiation.

75. The method of claim 1 wherein the patch further comprises packaging material.

76. The method of claim 1 wherein the patch releases the therapeutically effective amount of the essential oil over a period of time of up to about 12 hours.

77. The method of claim 1 wherein the patch releases the therapeutically effective amount of the essential oil over a period of time of up to about 8 hours.

78. The method of claim 1 wherein the source of the essential oil is located within about 6 inches of the mammal.

79. The method of claim 1 wherein the source of the essential oil is located within about 6 inches of nasal passageway of the mammal.

80. A method for preventing a respiratory viral infection in a mammal at risk thereof, the method comprises contacting a live respiratory virus with a prophylactically effective amount of an essential oil such that the live respiratory virus is inactivated upon contact with the essential oil, wherein the source of the essential oil is a patch located in the vicinity of the nasal passageway of the mammal.

81. A method for preventing the transmission of a respiratory infection between mammals, the method comprises contacting a live respiratory pathogen exiting the respiratory tract of a first mammal with a therapeutically effective amount of an essential oil such that the live respiratory pathogen is inactivated upon contact with the essential oil, wherein the source of the essential oil is a patch located in the vicinity of the nasal passageway of the first mammal.

82. A method for inhibiting a respiratory pathogen, the method comprises contacting a live respiratory pathogen with a therapeutically effective amount of an essential oil, such that the live respiratory pathogen is inactivated upon contact with the essential oil, wherein the source of the essential oil is a patch located in the vicinity of the respiratory pathogen.

83. A method for treating a respiratory infection in a mammal infected thereof or at risk thereof, the method comprises contacting a live respiratory pathogen with a therapeutically effective amount of an essential oil, such that the live respiratory pathogen is inactivated upon contact with the essential oil, wherein the source of the essential oil is a patch located in the vicinity of the nasal passageway of the mammal.

84. The method of claim 1, wherein the minimal inhibitory dose (MID) of the essential oil, as a measure of the vapor activity, is up to about 2.0 mg/L in air.

85. The method of claim 1, wherein the minimal inhibitory dose (MID) of the essential oil, as a measure of the vapor activity, is about 0.1 mg/L to about 2.0 mg/L in air.

86. A kit comprising:
(a) patch that comprises a flexible backing having a front side and a back side and a formulation positioned on at least a portion of the front side of the backing, in at least a portion of the front side of the backing, or on and in at least a portion of the front side of the backing, wherein the formulation comprises a prophylactically effective respiratory pathogen inhibiting amount of an essential oil;
(b) a mask for placing over the nasal passageway of a mammal; and
(c) packaging material.

87. The kit of claim 86 further comprising instructions for using the patch and mask.
